(19) United States
(12) Patent Application Publication
(10) Pub. No.: US 2022/0313347 A1
(43) Pub. Date: Oct. 6, 2022

(54) TISSUE ABLATION DEVICES AND METHODS

(71) Applicant: Innoblative Designs, Inc., Chicago, IL (US)

(72) Inventor: Robert F. Rioux, Ashland, MA (US)

(21) Appl. No.: 17/608,846

(22) PCT Filed: May 18, 2020
(86) PCT No.: PCT/US2020/033355

§ 371 Date: Nov. 4, 2021

Related U.S. Application Data
(60) Provisional application No. 62/850,316, filed on May 20, 2019.

(51) Int. Cl. A61B 18/14

(52) U.S. Cl. CPC A61B 18/14; A61B 2018/144; A61B 2018/1472; A61B 2018/1497; A61B 2018/1467; A61B 2018/00577

(57) ABSTRACT

The present invention is an ablation device having an arcuate configuration for the ablation of tissue. The device includes a probe having a nonconductive elongated shaft including at least one lumen therethrough and a nonconductive distal portion extending from the shaft. The nonconductive distal portion includes a plurality distal ports and a plurality of proximal ports in communication with the at least one lumen of the shaft. The device further includes an electrode array including a plurality of independent conductive wires extending through the lumen and positioned along an external surface of the nonconductive distal portion, each of the plurality of wires passes through at least an associated one of the proximal ports and through at least a corresponding one of the distal ports.

### CROSS-REFERENCE TO RELATED APPLICATIONS

This application claims the benefit of, and priority to, U.S. Provisional Application No. 62/850,316, filed May 20, 2019, the content of which is hereby incorporated by reference herein in its entirety. This application is also a continuation-in-part of U.S. Non-Provisional application Ser. No. 15/828,941, filed Dec. 1, 2017, which is a continuation of U.S. Non-Provisional application Ser. No. 15/624,327, filed Jun. 15, 2017 (now U.S. Pat. No. 9,839,472), which is a continuation of U.S. Non-Provisional application Ser. No. 15/337,334, filed Oct. 28, 2016 (now U.S. Pat. No. 9,848,936), which claims the benefit of, and priority to, U.S. Provisional Application No. 62/248,157, filed Oct. 29, 2015, and U.S. Provisional Application No. 62/275,984, filed Jan. 7, 2016, the contents of each of which are hereby incorporated by reference herein in their entirety.

### FIELD

The present disclosure relates generally to medical devices, and, more particularly, to tissue ablation devices configured to achieve coagulation, or hemostatic sealing, of a tissue.

### BACKGROUND

13% of the global population suffers from cancer and is expected to rise by approximately 70% over the next few decades based on statistics published by the World Health Organization (WHO). Many of the medical procedures related to cancer diagnosis and treatment require surgery to cut or carry soft tissue away. For example, during hepatic transection, one or more lobes of a liver containing abnormal tissue, such as malignant tissue or fibrous tissue caused by cirrhosis, are cut away. Regardless of the electrosurgical device used, extensive bleeding can occur, which can obstruct the surgeon's view and lead to dangerous blood loss levels, requiring transfusion of blood, which increases the complexity, time, and expense of the procedure, as well as the recovery time of the patient.

In order to prevent extensive bleeding or accumulation of fluid during surgery and to promote healing after surgery, hemostatic mechanisms, such as blood inflow occlusion, coagulants, as well as energy coagulation (e.g., electrosurgical coagulation or argon-beam coagulation) can be used. Unlike resection, which involves application of highly intense and localized heating sufficient enough to break intercellular bonds, energy coagulation of tissue involves the application of low level current that denatures cells to a sufficient depth without breaking intercellular bonds, i.e., without cutting the tissue.

However, current energy coagulation devices include needle-like devices, which can only generate small regions of heat, requiring frequent repositioning of the device in order to treat larger areas. As such, great care must be taken when using such energy coagulation devices in order to reduce charring of tissue, avoid tissue from becoming stuck on the device, and most importantly, to minimize the increase of tissue resistance, so as to avoid reducing the efficiency of the overall procedure. Unfortunately, regardless of the care taken, the current energy coagulation modalities result in long procedure times and variable ablation depths, making it nearly impossible to maintain hemostasis at the treatment site.

### SUMMARY

The present invention relates to ablation devices configured to destroy large surface areas of tissue in a consistent manner in an effort to achieve coagulation. The systems and methods described herein can be used during a resection procedure to coagulate cross-sectional tissue of the resection site so as to prevent or stop fluid accumulation (e.g., blood from vessel(s)) as a result of the resection of tissue. Accordingly, the ablation device of the present invention may be particularly useful during or after procedures involving the removal of unhealthy, or otherwise undesired, tissue from any part of the body. Thus, tumors, both benign and malignant, may be removed via a surgical intervention, and the ablation devices described herein can then be used to coagulate the tissue of the resection site. The ablation devices are configured to be applied to tissue in a sweeping motion, thus allowing for real-time assessment of progress of ablation, without needing to pause to assess the progress.

In particular, the present disclosure is generally directed to a tissue ablation system including an ablation device to be used on large surface areas of tissue and emit non-ionizing radiation, such as radiofrequency (RF) energy, to treat surface lesions. The tissue ablation device of the present invention generally includes a probe including an elongated shaft configured as a handle and adapted for manual manipulation and a nonconductive distal portion coupled to the shaft. The nonconductive distal portion is a rigid arcuate body and includes an electrode array positioned along the arcuate body's convex exterior surface. The distal portion, including the electrode array, can be delivered to a resection site and the convex side of the distal portion can be swept along the surface of cross-sectional tissue (e.g., tissue remaining after tumor removal) and configured to coagulate the tissue (via RF energy) in contact therewith.

In one aspect, the electrode array is composed of a plurality of conductive members (e.g., conductive wires) electrically isolated and independent from one another. Thus, in some embodiments, each of the plurality of conductive wires, or one or more sets of a combination of conductive wires, is configured to independently receive an electrical current from an energy source (e.g., ablation generator) and independently conduct energy, the energy including RF energy. This allows energy to be selectively delivered to a designated conductive wire or combination of conductive wires. This design also enables the ablation device to function in a bipolar mode because a first conductive wire (or combination of conductive wires) can deliver energy to the surrounding tissue through its electrical connection with an ablation generator while a second conductive wire (or combination of conductive wires) can function as a ground or neutral conductive member.

The independent control of each wire or sets of wires allows for activation (e.g., emission of RF energy) of corresponding portions of the electrode array. For example, the electrode array may be partitioned into specific portions which may correspond to clinical axes or sides of the distal portion of the device. In one embodiment, the electrode array may include at least two distinct portions (i.e., individual or sets of conductive wires) corresponding to one side of the distal portion (e.g., the convex side or two quadrants of the arcuate body).

The ablation device is configured to provide RF ablation via a virtual electrode arrangement, which includes distribution of a fluid along a convex exterior surface of the hemispherical body of the distal tip and, upon activation of the electrode array, the fluid may carry, or otherwise promote, energy emitted from the electrode array to the surrounding tissue. For example, the nonconductive distal portion of the ablation device includes an interior chamber retaining at least a hydrophilic insert. The interior chamber of the distal portion is configured to receive and retain a fluid (e.g., saline) therein from a fluid source. The hydrophilic insert is configured to receive and evenly distribute the fluid through the distal tip by wicking the saline against gravity. The distal portion may generally include a plurality of ports or apertures configured to allow the fluid to pass therethrough, or weep, from the interior chamber to a convex exterior surface of the distal portion.

In some embodiments, the device is configured so that a constant flow of fluid is not needed. For example, the distal tip may be defined by a first halve having at least a concave portion and a second halve having at least a convex portion complementary to the concave portion of the first halve, wherein the halves are nested together to form a rigid arcuate body having at least an exterior cavity and an interior chamber. The exterior cavity may be configured to receive fluid from an external fluid source. The external fluid source may be any type of container containing the fluid, such as a bottle or bowl and the fluid may be poured directly into the cavity of the device from the container. The exterior cavity may be configured to have a plurality of receiving ports. The receiving ports may be configured to pass the fluid from the exterior cavity into the interior chamber of the hemispherical body containing the hydrophilic insert. The hydrophilic insert is shaped and sized to maintain sufficient contact with the surface of the interior chamber of the distal tip wall, and specifically in contact with one or more fluid receiving ports and one or more ports of the convex side of the arcuate body to uniformly distribute the fluid to the ports of the convex side.

In some other embodiments, the device is configured to receive fluid from an internal fluid source. For example, the hemispherical body of the distal tip may be defined by a first halve having at least a solid and relatively planar portion and a second halve having at least a convex portion, the halves are coupled together to form a rigid arcuate body having at least an interior chamber retaining at least a spacer member and the hydrophilic insert. In some embodiments, the spacer member is shaped and sized so as to maintain the hydrophilic insert in contact with the interior surface of the distal tip wall, and specifically in contact with the one or more perforations on the convex portion of the arcuate body, such that the hydrophilic insert provides uniformity of saline distribution to the perforations.

It should be noted that, in some embodiments, the arcuate body is generally in the form of a hemispherical shape, while in other embodiments, the arcuate body may include a hemi ellipsoidal shape or a hemiovoidal shape.

Accordingly, upon positioning the convex side of the distal portion within a target site (e.g., tissue to be ablated), the electrode array can be activated. The fluid weeping through the perforations to the convex outer surface of the distal portion is able to carry energy from electrode array, thereby creating a virtual electrode. Accordingly, upon the fluid weeping through the perforations, a pool or thin film of fluid is formed on the convex exterior surface of the distal portion and is configured to ablate surrounding tissue via the RF energy carried from the electrode array.

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIG. 1 is a schematic illustration of an ablation system consistent with the present disclosure;

FIG. 2 is a perspective view of an ablation device tip of the ablation system of FIG. 1;

FIGS. 3A, 3B, and 3C are perspective views of the ablation device tip of FIG. 2 in greater detail;

FIGS. 4A and 4B schematically illustrate the ablation device tip of the ablation device of FIG. 1 with and without the nonconductive distal portion, respectively;

FIG. 5A is a side view and FIG. 5B is a cross-sectional view of one embodiment of a deployable distal portion of the ablation device illustrating transitioning of the distal portion from a delivery configuration to a deployed configuration;

FIG. 6 illustrates a method of deploying the distal portion of FIGS. 5A and 5B into an expanded configuration for delivery of RF energy to a target site for ablation of marginal tissue;

FIG. 7 is a cross-sectional view of the deployable distal portion of FIGS. 5A and 5B illustrating the inclusion of an internal balloon member within an interior chamber of the distal portion. The internal balloon is configured to receive a fluid from a fluid source and thereby expand, which, in turn, causes the distal portion to transition from the delivery configuration to the deployed configuration, and further supply the fluid to an exterior surface of the distal portion, via weeping of the fluid through one or more perforations on the distal portion wall, to create a virtual electrode arrangement with the electrode array;

FIG. 8 is an exploded view of an ablation device consistent with the present disclosure, including a hydrophilic insert provided within an interior chamber of the distal portion and configured to receive a fluid from a fluid source and evenly distribute the fluid to an exterior an exterior surface of the distal portion, via weeping of the fluid through one or more perforations on the distal portion wall, to create a virtual electrode arrangement with the electrode array;

FIG. 9 is an exploded view of the ablation device of FIG. 8 illustrating the hydrophilic insert in more detail;

FIGS. 10A-10E are perspective views of a distal tip of the ablation device of FIG. 1 illustrating various electrode array configurations;

FIG. 11 is a side view of the distal tip of the ablation device of FIG. 1 including several clinical axes or sides. Each clinical axis or side includes one or more independently connected electrodes, which enables differential function and current independent drives and/or measurements;

FIGS. 12A, 12B, 12C, and 12D are side and perspective views of the distal tip of the application device illustrating the different clinical axes or sides of FIG. 11;

FIGS. 13 and 14 are perspective and exploded perspective views, respectively, of one embodiment of a device controller consistent with the present disclosure;

FIG. 15 is an exploded perspective view of another embodiment of an ablation device consistent with the present disclosure;

FIG. 16 is a plan view of the ablation device of FIG. 15 illustrating the two halves of the device separated from one another and showing the external surface of each;

FIG. 17 is a plan view of the ablation device of FIG. 15 illustrating the two halves of the device separated from one another and showing the interior surface of each;

FIGS. 18A and 18B are enlarged views of the spheroid body of the first halve of the device showing the exterior and interior surfaces, respectively, and further illustrating the particular arrangement of first and second conductive wires extending through proximal and distal ports of the spheroid body;

FIGS. 19A and 19B are enlarged views of the spheroid body of the second halve of the device showing the exterior and interior surfaces, respectively, and further illustrating the particular arrangement of third and fourth conductive wires extending through proximal and distal ports of the spheroid body;

FIG. 20 is a schematic illustration of the ablation device of FIG. 15 illustrating delivery of fluid from the irrigation pump, as controlled by the controller, to the hydrophilic insert within the interior chamber of the distal portion of the device, wherein the fluid can be subsequently distributed to an exterior surface of the distal portion resulting in a virtual electrode arrangement upon activation of one or more portions of the electrode array;

FIG. 21 is a perspective view of a detachable mount for holding a temperature probe (or any other separate monitoring device) at a desired position relative to the distal portion of the ablation device for the collection of temperature data during an RF ablation procedure;

FIG. 22 is a plan view of the detachable mount holding the temperature probe relative to the distal portion of the ablation device;

FIG. 23 is a perspective view of another embodiment of an ablation device consistent with the present disclosure;

FIG. 24 is an exploded perspective view of the ablation device of FIG. 23;

FIG. 25 is a plan view of an ablation device of FIG. 23 illustrating the two halves of the device separated from one another and showing the interior surface of each;

FIG. 26 is a plan view of an ablation device of FIG. 23 illustrating the two halves of the device separated from one another and showing the external surface of each;

FIG. 27 is perspective view of another embodiment of an ablation device consistent with the present disclosure;

FIG. 28 is an exploded perspective view of the ablation device of FIG. 27;

FIG. 29 is a cross-sectional view of the hemispherical body of the ablation device of FIG. 27 illustrating the interior of the hemispherical body;

FIG. 30 is a plan view of the ablation device of FIG. 27 illustrating the two halves of the device separated from one another and showing the external surface of each; and

FIG. 31 is a perspective view of the ablation device of FIG. 27 illustrating receipt of the conductive fluid from an external source.

FIGS. 32A and 32B are top and side views, respectively, of an exemplary shape of the distal portion of the device, illustrating a hemispherical shape.

FIGS. 33A and 33B are top and side views, respectively, of another exemplary shape of the distal portion of the device, illustrating a hemiellipsoidal shape.

FIGS. 34A and 34B are top and side views, respectively, of one exemplary shape of the distal portion of the device, illustrating a hemiovoidal shape.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present disclosure is generally directed to a tissue ablation system including an ablation device to be delivered to a target site to achieve coagulation of tissue. In some aspects, ablation devices may have an arcuate rigid head configured to be applied to tissue and to ablate large surface areas of tissue, and in other aspects ablation devices with a deployable applicator head are configured to be applied to tissue and delivered into a tissue cavity and ablate marginal tissue surrounding the tissue cavity.

A tissue ablation system consistent with the present disclosure may be well suited for treating larger surface areas of tissue, such as the cross-section of a resected liver lobe. Accordingly, the ablation device of the present invention may be particularly useful during procedures involving the removal of unhealthy, or otherwise undesired, tissue from any part of the body. Thus, tumors, both benign and malignant, may be removed via a surgical intervention and the ablation devices described herein can be used to coagulate the tissue of the resection site. The tissue system of the present disclosure can also be used on large surface areas of tissue to treat surface lesions.

In particular, the present disclosure is generally directed to a surface tissue ablation system including an ablation device to be delivered to the surface of tissue to emit non-ionizing radiation, such as radiofrequency (RF) energy, in a desired shape or pattern so as to deliver treatment for the ablation and coagulation of large surface areas of tissue.

The tissue ablation device of the present invention generally includes a probe including an elongated shaft configured as a handle and adapted for manual manipulation and a nonconductive distal portion coupled to the shaft. The nonconductive distal portion is a rigid arcuate body and includes an electrode array positioned along a convex exterior surface of the body. The distal portion, including the electrode array, can be delivered to a resection site and the convex side of the distal portion can be swept along the surface of cross-sectional tissue (e.g., tissue remaining after tumor removal) and configured to coagulate the tissue (via RF energy) in contact therewith.

The tissue ablation device of the present disclosure is configured to allow surgeons, or other medical professionals, to deliver controlled doses of RF energy at consistent depths to large surface areas of tissue to achieve coagulation in a quick and efficient manner. Importantly, the devices are configured to be applied to tissue in a sweeping motion, thus allowing for real-time assessment of progress of ablation, without the need to stop treatment to assess the progress. By using a sweeping or painting technique, the device is able to cover large surface areas of tissue in a short amount of time. For example, after liver resection, the device may be used to paint the entire cross-section of a resected liver lobe without pausing to assess the progress of the ablation.

In other aspects, a tissue ablation system consistent with the present disclosure may also be well suited for treating hollow body cavities, such as irregularly-shaped cavities in breast tissue created by a lumpectomy procedure. For example, once a tumor has been removed, a tissue cavity remains. The tissue surrounding this cavity is the location within a patient where a reoccurrence of the tumor may most likely occur. Consequently, after a tumor has been removed, it is desirable to destroy the surrounding tissue (also referred herein as the “margin tissue” or “marginal tissue”).

The tissue ablation system of the present disclosure can be used during an ablation procedure to destroy the thin rim of marginal tissue around the cavity in a targeted manner. In particular, the present disclosure is generally directed to a cavitary tissue ablation system including an ablation device to be delivered into a tissue cavity and configured to emit non-ionizing radiation, such as radiofrequency (RF) energy, in a desired shape or pattern so as to deliver treatment for the ablation and destruction of a targeted portion of marginal tissue around the tissue cavity.

The tissue ablation device of the present invention generally includes a probe including an elongated shaft configured as a handle and adapted for manual manipulation and a nonconductive distal portion coupled to the shaft. The nonconductive distal portion includes an electrode array positioned along an external surface thereof. The distal portion, including the electrode array, can be delivered to and maneuvered within a tissue cavity (e.g., formed from tumor removal) and configured to ablate marginal tissue (via RF energy) immediately surrounding the tissue cavity in order to minimize recurrence of the tumor. The tissue ablation device of the present disclosure is configured to allow surgeons, or other medical professionals, to deliver precise, measured doses of RF energy at controlled depths to the marginal tissue surrounding the cavity.

Accordingly, a tissue ablation device consistent with the present disclosure may be well suited for treating hollow body cavities, such as irregularly-shaped cavities in breast tissue created by a lumpectomy procedure. It should be noted, however, that the devices of the present disclosure are not limited to such post-surgical treatments and, as used herein, the phrase “body cavity” may include non-surgically created cavities, such as natural body cavities and passages, such as the ureter (e.g. for prostate treatment), the uterus (e.g. for uterine ablation or fibroid treatment), fallopian tubes (e.g. for sterilization), and the like. Additionally, or alternatively, tissue ablation devices of the present disclosure may be used for the ablation of marginal tissue in various parts of the body and organs (e.g., skin, lungs, liver, pancreas, etc.) and is not limited to treatment of breast cancer.

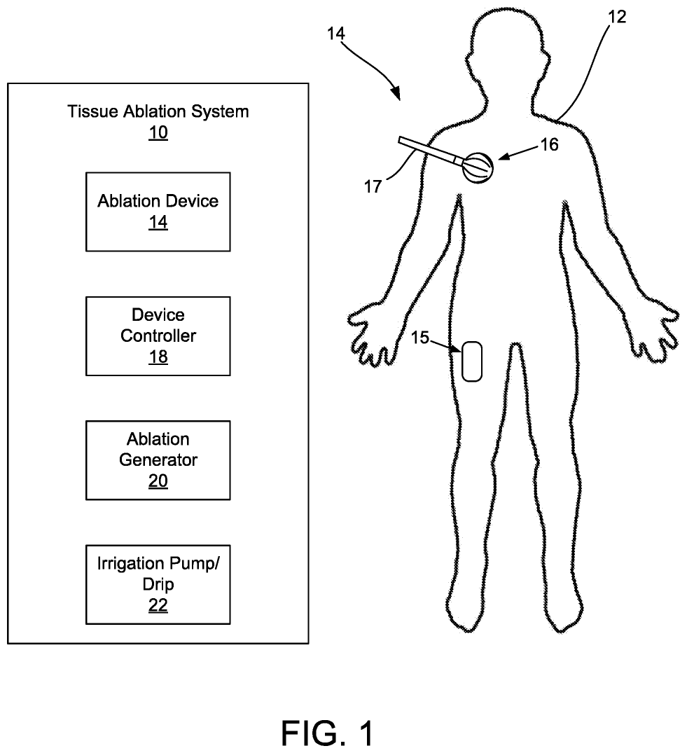

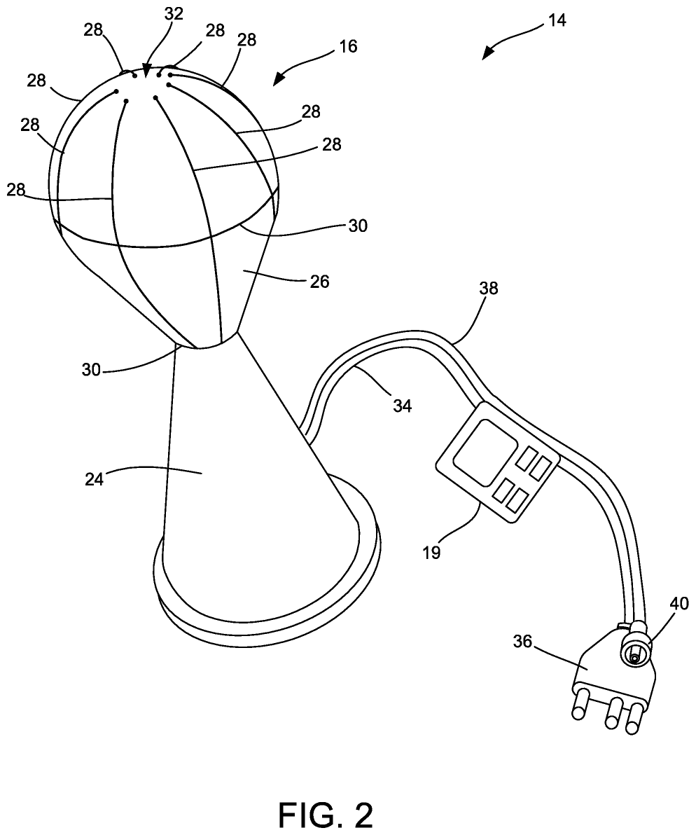

FIG. 1 is a schematic illustration of an ablation system 10 for providing targeted ablation of marginal tissue during a tumor removal procedure in a patient 12. The ablation system 10 generally includes an ablation device 14, which includes a probe having a distal tip or portion 16 and an elongated catheter shaft 17 to which the distal tip 16 is connected. The catheter shaft 17 may generally include a nonconductive elongated member including a fluid delivery lumen. The ablation device 14 may further be coupled to a device controller 18 and an ablation generator 20 over an electrical connection (electrical line 34 shown in FIG. 2), and an irrigation pump or drip 22 over a fluid connection (fluid line 38 shown in FIG. 2). The ablation generator 20 may also connected to a return electrode 15 that is attached to the skin of the patient 12.

As will be described in greater detail herein, the device controller 18 may be used to control the emission of energy from one or more conductive members of the device 14 to result in ablation. In other embodiments, the device controller 18 may also control the delivery of fluid to the applicator head 16 so as to control subsequent weeping of fluid from the head 16 during an RF ablation procedure. In some embodiments, the applicator head 16 may receive the fluid from an external source, such as by pouring the fluid from a container into the head 16. In some cases, the device controller 18 may be housed within the ablation device 14. The ablation generator 20 may also be connected to a return electrode 15 that is attached to the skin of the patient 12.

As will be described in greater detail herein, during an ablation treatment, the ablation generator 20 may generally provide RF energy (e.g., electrical energy in the radiofrequency (RF) range (e.g., 350-800 kHz)) to an electrode array of the ablation device 14, as controlled by the device controller 18. At the same time, saline may also be released from the head 16. The RF energy travels through the blood and tissue of the patient 12 to the return electrode 15 and, in the process, ablates the region(s) of tissues adjacent to portions of the electrode array that have been activated.

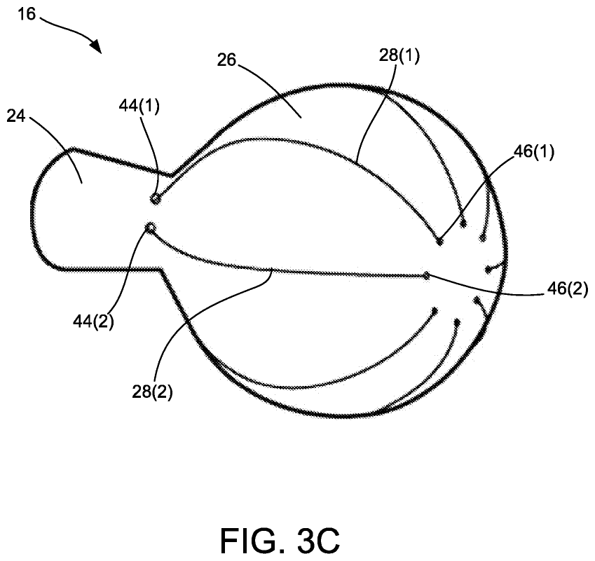

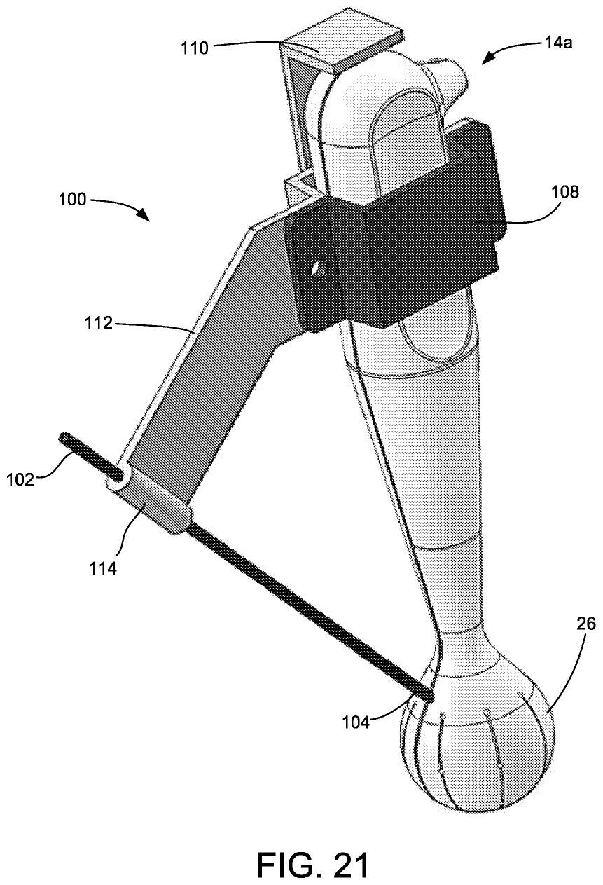

FIG. 2 is a perspective view of the distal portion or tip 16 of the ablation device 14. The distal tip 16 may include a neck portion 24 and a generally spheroid body 26 extending distally from the neck 24. In some embodiments, the spheroid body 26, may only be one-half, that may generally be in the form of a hemispherical shape, as will be described in greater detail herein, particularly with respect to FIGS. 23, 24, 25, 26, 27A, 27B, 28, 29, 30, and 31. It should be noted that, in some embodiments, the spheroid body 26 may be generally rigid and may maintain a default shape. However, in some embodiments, the spheroid body 26 may be configured to transition between a collapsed state and an expanded state, as will be described in greater detail herein, particular with respect to FIGS. 5A-5B and 6-7. For example, the spheroid body 26 may be collapsible to a delivery configuration having a reduced size (e.g., equatorial diameter) relative to the deployed configuration size (e.g., equatorial diameter) of the spheroid body 26.

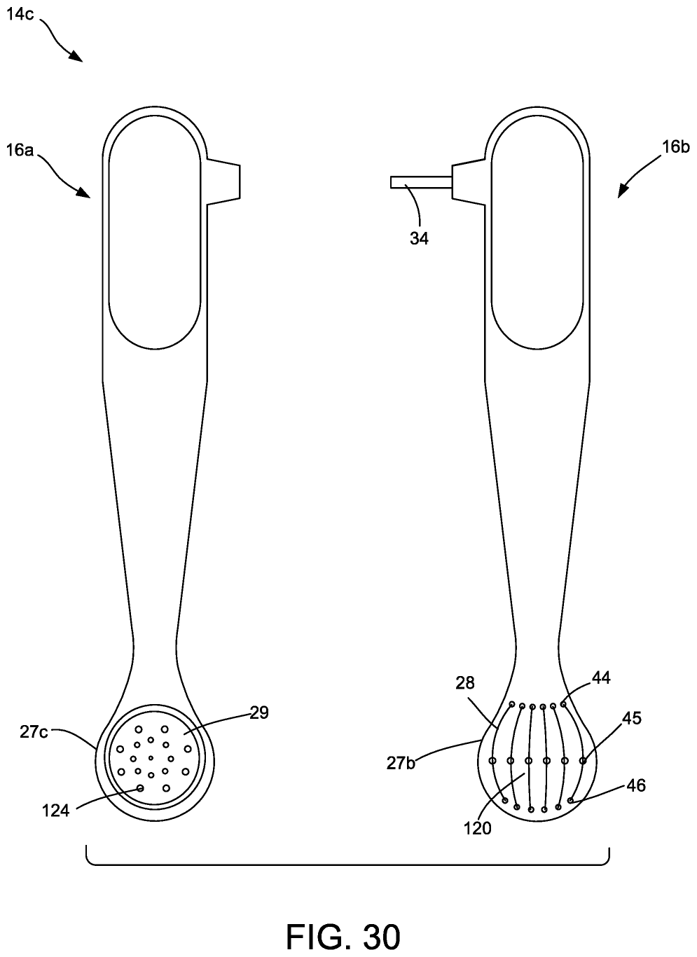

It should further be noted that the distal portion or tip 16, specifically body 26 is not limited to a spheroid or hemispherical shape. Rather, the distal portion or tip 16 consistent with the present disclosure described herein, can be in the form of various arcuate shapes. For example, the body 26 may include a spherical shape, including, but not limited to, an oblate spheroid, a prolate spheroid, and various forms therebetween. The body 26 may also include other arcuate shapes, such as an elliptical shape, an oval shape, and various forms thereof, as will be described in greater detail herein, particularly with regard to FIGS. 32A, 32B, 33A, 33B, 34A, and 34B. For sake of consistency, the following description refers to the body 26, 27 as being spheroid or hemispherical, respectively.

In some examples, the spheroid body 26 includes a non-conductive material (e.g., a polyamide) as a layer on at least a portion of an internal surface, an external surface, or both an external and internal surface. In other examples, the spheroid body 26 is formed from a non-conductive material. Additionally or alternatively, the spheroid body 26 material can include an elastomeric material or a shape memory material.

In some examples, the spheroid body 26 or the hemispherical body (e.g., as illustrated in FIG. 23) has a diameter (e.g., an equatorial diameter) of about 80 mm or less. In certain implementations, the spheroid body 26 of the distal tip, in a deployed configuration, has an equatorial diameter of 2.0 mm to 60 mm (e.g., 5 mm, 10 mm, 12 mm, 16 mm, 25 mm, 30 mm, 35 mm, 40 mm, 50 mm, and 60 mm). Based on the surgical procedure, the collapsibility of the spheroid body 26 can enable the distal tip to be delivered using standard sheaths (e.g., an 8F introducer sheath). However, the spheroid body 26 need not be collapsible in some procedures, and thus has a relatively rigid body and maintains the default shape.

The distal tip 16 of the ablation device 14 further includes an electrode array positioned thereon. The electrode array includes at least one conductive member 28. As illustrated in the figures, the electrode array may include at least eight conductive members 28. Accordingly, the electrode array may include a plurality of conductive members 28. The plurality of conductive members 28 extend within the distal tip 16, through a channel 32 and along an external surface of the spheroid body 26. The conductive members 28 extend along the longitudinal length of the distal tip 16 and are radially spaced apart (e.g., equidistantly spaced apart) from each other. These conductive members transmit RF energy from the ablation generator and can be formed of any suitable conductive material (e.g., a metal such as stainless steel, nitinol, or aluminum). In some examples, the conductive members 28 are metal wires. Accordingly, for ease of description, the conductive member(s) will be referred to hereinafter as “conductive wire(s) 28”.

As illustrated, one or more of the conductive wires 28 can be electrically isolated from one or more of the remaining conductive wires 28. This electrical isolation enables various operation modes for the ablation device 14. For example, ablation energy may be supplied to one or more conductive wires 28 in a bipolar mode, a unipolar mode, or a combination bipolar and unipolar mode. In the unipolar mode, ablation energy is delivered between one or more conductive wires 28 on the ablation device 14 and the return electrode 15, as described with reference to FIG. 1. In bipolar mode, energy is delivered between at least two of the conductive wires 28, while at least one conductive wire 28 remains neutral. In other words, at least, one conductive wire functions as a grounded conductive wire (e.g., electrode) by not delivering energy over at least one conductive wire 28.

The electrode array may further include one or more stabilizing members 30 configured to provide support for the plurality of conductive wires 28. The one or more stabilizing member 30 generally extend along a surface (e.g., external or internal) of the distal tip 16 so as to circumscribe the spheroid body 26. The stabilizing members 30 can, in some examples, electrically connect to one or more conductive wires 28. In other examples, the stabilizing members 30 are non-conductive. The stabilizing members 30 can be formed of a suitably stiff material (e.g., metal such as stainless steel, nitinol, or aluminum). In some implementations, the stabilizing members 30 can be integral with a portion of the spheroid body 26 (e.g., as a rib). While, the distal tip 16 is generally shown with one or more stabilizing members, in some implementations, the distal tip 16 is free of stabilizing members.

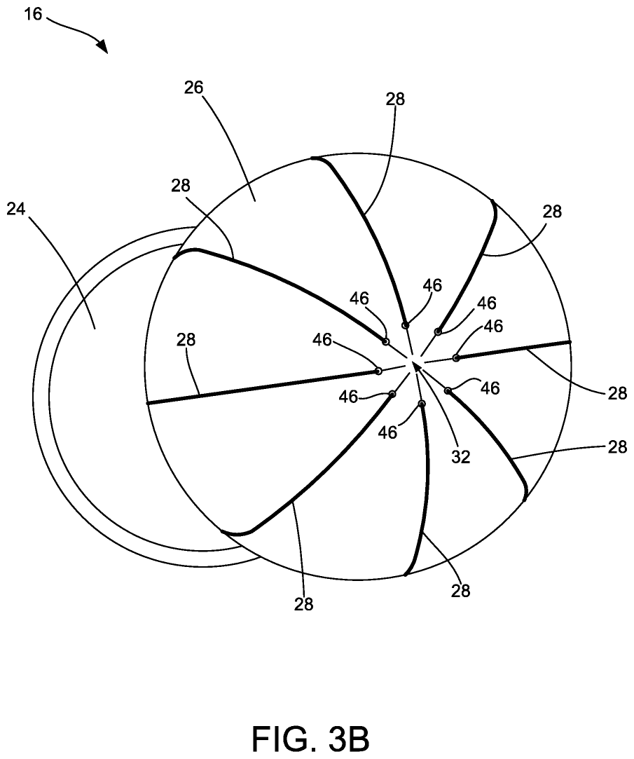

To further aid in illustrating the arrangement of the conductive wires 28 and the non-conductive spheroid body 26, FIG. 4A shows the conductive wires 28 positioned over the non-conductive spheroid body 26 while FIG. 4B shows the electrode array of the ablation device without the non-conductive spheroid body 26.

As shown, the distal tip 16 may be coupled to the ablation generator 20 and/or irrigation pump 22 via an electrical line 34 and a fluid line 38, respectively. Each of the electrical line 34 and fluid line 38 may include an adaptor end 36, 40, respectively, configured to couple the associated lines with a respective interface on the ablation generator 20 and irrigation pump 22. In some examples, the ablation device 14 may further include a user switch or interface 19 which may serve as the device controller 18 and thus, may be in electrical communication with the ablation generator 20 and the ablation device 14, as well as the irrigation pump 22 for controlling the amount of fluid to be delivered to the tip 16.

The switch 19 can provide a user with various options with respect to controlling the ablation output of the device 14, as will be described in greater detail herein. For example, the switch 19, which may serve as the device controller 18, may include a timer circuit, or the like, to enable the conductive wires 28 to be energized for a pre-selected or desired amount of time. After the pre-selected or desired amount of time elapses, the electrical connection can be automatically terminated to stop energy delivery to the patient. In some cases, the switch 19 may be connected to individual conductive wires 28. For example, in some embodiments, the switch 19 may be configured to control energy delivery from the ablation generator 20 so that one or more individual conductive wires, or a designated combination of conductive wires, are energized for a pre-selected, or desired, duration.

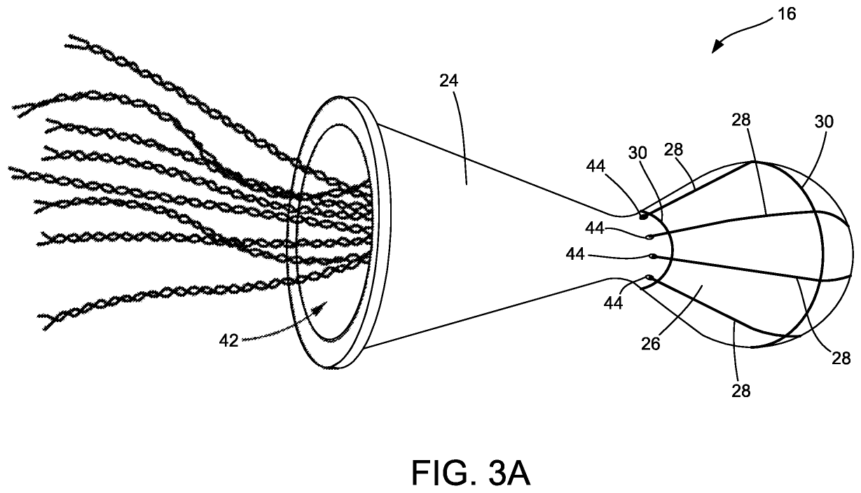

FIGS. 3A, 3B, and 3C are perspective views of the distal tip 16 of FIG. 2 in greater detail. As shown in FIGS. 2 and 3A-3C, the conductive wires 28 extend through a lumen 42 within the distal tip 16. For example, each of the conductive wires 28 enters the lumen 42 of the neck 24 and extends through the distal tip portion 16 before exiting the distal tip through either a center channel 32 at a distal most portion of the distal tip or one of a plurality of proximal ports 44. In some examples, a plurality of distal ports 46 extending through a wall of the distal tip 16 is positioned around the channel 32. A plurality of proximal ports 44 can also extend through a wall of the distal tip 16. These proximal ports 44 can be positioned around the distal tip 16 in close proximity (e.g., within at least 5 mm, within at least 3 mm, within at least 1 mm, within 0.5 mm, within 0.4 mm, or within 0.2 mm) to the junction between the spheroidal body 26 and the neck 24 of the distal tip 16. In some cases, the number of proximal ports 44 and distal ports 46 is equal to the number of conductive wires 28.

In some examples, each conductive wire 28 can extend through a different distal port 46, which allows the conductive wires 28 to remain electrically isolated from one another. In other examples, one or more conductive wires can extend through the same distal port 46.

Upon passing through a distal port 46, each conductive wire 28 can extend along an external surface of the distal tip 16. In some examples, the length of the conductive wire 28 extending along the external surface is at least 20% (e.g., at least, 50%, 60%, 75%, 85%, 90%, or 99%) of the length of the spheroid body 26. The conductive wire 28 can then re-enter the lumen 42 of the distal tip 16 through a corresponding proximal port 44. For example, as shown in FIG. 3C, conductive wire 28(1) passes through distal port 46(1), extends along a length of the external surface of the distal tip 16, and passes through an associated proximal port 44(1) into the lumen 42 of the distal tip 16, while conductive wire 28(2) is electrically isolated from conductive wire 28(1) in that it passes through associated proximal and distal ports 44(2), 46(2), respectively.

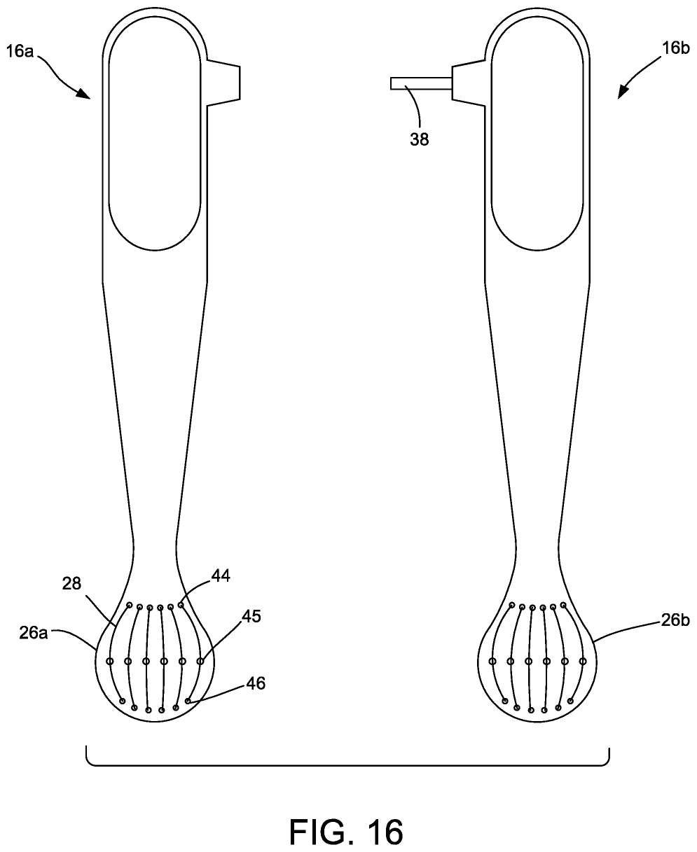

In some examples, each conductive wire 28 can extend through a different associated proximal port 44, which allows the conductive wires 28 to remain electrically isolated from one another. In other examples, one or more conductive wires can extend through the same proximal port. Yet still, as will be described in greater detail herein, particularly with reference to the devices 14a, 14b and 14c illustrated in FIGS. 18A-18B, 19A-19B and 27A-27B, an individual conductive wire can extend through multiple proximal and distal ports.

In some embodiments, the spheroid body 26 may be configured to transition between a collapsed state and an expanded state, which may allow for a surgeon to introduce the distal portion 26 into certain areas of the body that may have reduced openings and could be difficult to access with when the spheroid body is in the default shape. FIG. 5A is a side view and FIG. 5B is a cross-sectional view of one embodiment of a deployable distal portion 26 of the ablation device 14 illustrating transitioning of the distal portion 26 from a delivery configuration to a deployed configuration.

As shown, when in a delivery configuration, the spheroid body 26 may generally have a prolate-spheroid shape, thereby having a reduced size (e.g., equatorial diameter) relative to the deployed configuration size (e.g., equatorial diameter). In some embodiments, the spheroid body 26 may be configured to transition between the delivery and deployed configurations via manipulation of one or more of the conductive wires. For example, as shown in FIG. 5B, at least one conductive wire 28 may be configured to translate axially along a longitudinal axis of the distal tip 16, which, in turn, can exert a force on at least a region of the distal tip 16 that causes or partially causes the spheroid body 26 to transition or deform between a delivery configuration to a deployed configuration. For example, axial translation of the conductive wire 28 along an axial direction exerts a force on the distal tip 16, which causes spheroid body 26 to assume a more spherical configuration for deployment. In other words, axially translating the conductive wire 28 causes the spheroid body 26 to transition from a delivery configuration in which the spheroid body 26 exhibits a prolate-spheroid shape to a deployment configuration in which the spheroid body 26 exhibits an oblate-spheroid shape.

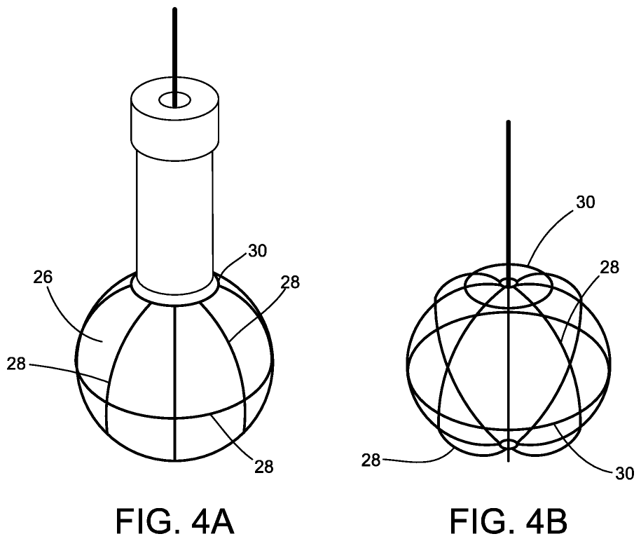

FIG. 6 illustrates a method of deploying the distal portion 16 into an expanded configuration for delivery of RF energy to a target site for ablation of marginal tissue. As shown, the catheter shaft 17 of the ablation device 14 can optionally include a dedicated control wire connected to a knob or control mechanism accessible on the catheter shaft. In this example, one or more control wires or other components may be coupled to the conductive wires to control the retraction and expansion (e.g., via pushing along direction 48 and pulling along direction 50) of the distal tip 105 from the catheter shaft 107. In addition, other components (e.g., electrical wiring for electrically coupling the conductive element and RF generator) can also be housed within the, at least, one lumen of the catheter shaft 17 of the ablation device 14.

In some implementations, the catheter shaft 17 can be configured as a handle adapted for manual manipulation. In some examples, the catheter shaft 17 is additionally or alternatively configured for connection to and/or interface with a surgical robot, such as the Da Vinci® surgical robot available from Intuitive Surgical, Inc., Sunnyvale, Calif. The catheter shaft 17 may be configured to be held in place by a shape lock or other deployment and suspension system of the type that is anchored to a patient bed and which holds the device in place while the ablation or other procedure takes place, eliminating the need for a user to manually hold the device for the duration of the treatment.

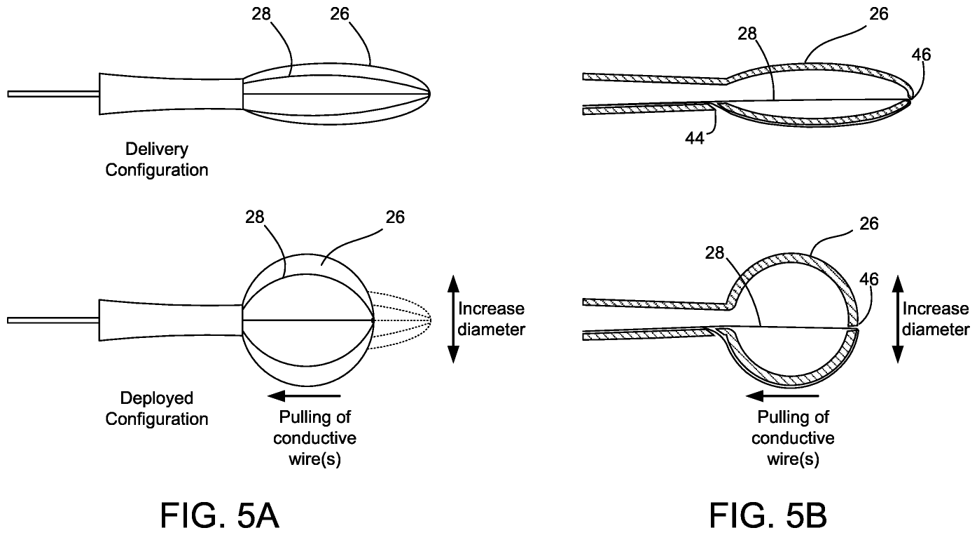

FIG. 7 is a cross-sectional view of the deployable distal portion of FIGS. 5A and 5B illustrating the inclusion of an internal balloon member within an interior chamber of the distal portion 26. The internal balloon is configured to receive a fluid from a fluid source and thereby expand, which, in turn, causes the distal portion to transition from the delivery configuration to the deployed configuration, and further supply the fluid to an exterior surface of the distal portion, via weeping of the fluid through one or more perforations on the distal portion wall (e.g., distal port 46), to create a virtual electrode arrangement with the electrode array. For example, the inner balloon may include a plurality of perforations, holes, or micropores in the balloon wall so as to allow a fluid provided within the balloon (e.g., saline) to pass therethrough, or weep, from the balloon when the balloon is inflated. The perforations may be sized, shaped, and/or arranged in such a pattern so as to allow a volume of fluid to pass from the interior volume of the balloon into the interior chamber of the distal portion 26 and then to pass through one or more perforations, holes, or micropores formed in the distal portion wall to an exterior surface of the tip at a controlled rate so as to allow the balloon to remain inflated and maintain its shape.

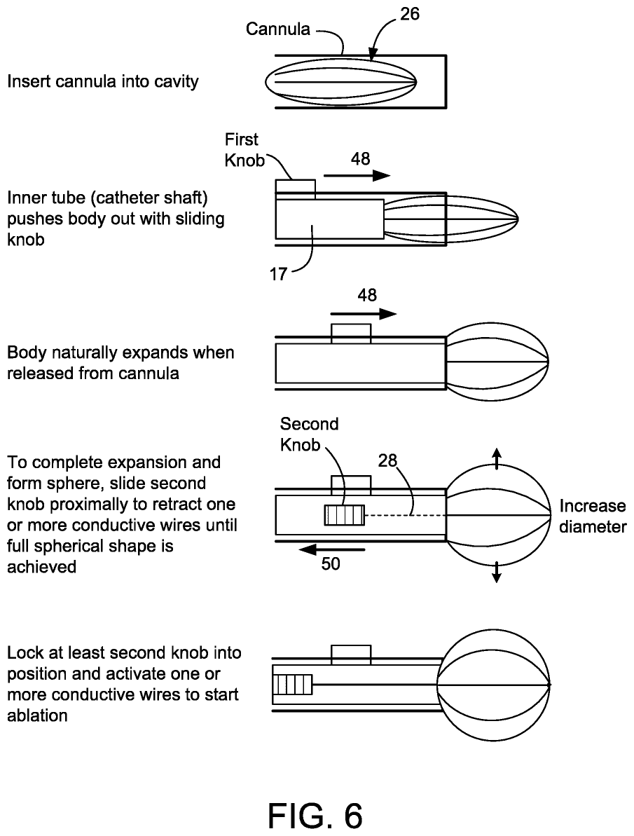

FIG. 8 is an exploded view of an ablation device 14 consistent with the present disclosure. As shown, in some implementations, the ablation device 14, specifically the distal tip 16, may be formed from two or more pieces (tip halves 16a and 16b) configured to be coupled to one another to form the unitary distal tip 16. Each half 16a and 16b includes cooperating neck portions 24a, 24b and spheroid bodies 26a, 26b, as well as a cap 52 to be coupled to both halves 16a and 16b so as to fully enclose the interior of the distal tip 16. As further illustrated, an electrical line 34 may be provided for coupling the conductive wires 28 to the controller 18 and ablation generator 20 and a fluid line 38 may be provided for providing a fluid connection between the irrigation pump or drip 22 to the distal tip 16 so as to provide a conductive fluid (e.g., saline) to the tip 16. The electrical line 34 and/or the fluid delivery line 38 can be supported by a stabilizing element 62 within the device lumen. In some cases, the stabilizing element 62 may be integral with the neck 24 of the distal tip 16.

As previously described, conductive members 28 extend through a first port (e.g., the distal port 44), run along an external surface of the spheroid body 26 (e.g. within the groove 47) before re-entering the lumen of the distal tip 16 through another port (e.g., the proximal port 46). A conductive fluid, such as saline, may be provided to the distal tip 16 via the fluid line 38, wherein the saline may be distributed through the ports (e.g., to the distal ports 44, the proximal ports 46, and/or medial ports 45). The saline weeping through the ports and to an outer surface of the distal tip 16 is able to carry electrical current from electrode array, such that energy is promoted from the electrode array to the tissue by way of the saline weeping from the ports, thereby creating a virtual electrode. Accordingly, upon the fluid weeping through the ports, a pool or thin film of fluid is formed on the exterior surface of the distal tip 16 and is configured to ablate surrounding tissue via the electrical current carried from the electrode array.

As shown, the ablation device 14 may further include hydrophilic insert 54 aligned with the fluid delivery line 38 and positioned within the interior chamber formed between the two halves 16a, 16b. The hydrophilic insert 54 is configured to distribute fluid (e.g., saline) delivered from the fluid line 38 through the distal tip 16 by, for example, wicking the saline against gravity. This wicking action improves the uniformity of saline distribution to the device ports (e.g., to the proximal ports 44, the distal ports 46, and/or a medial ports 45). The hydrophilic insert 54 can be formed from a hydrophilic foam material (e.g., hydrophilic polyurethane).

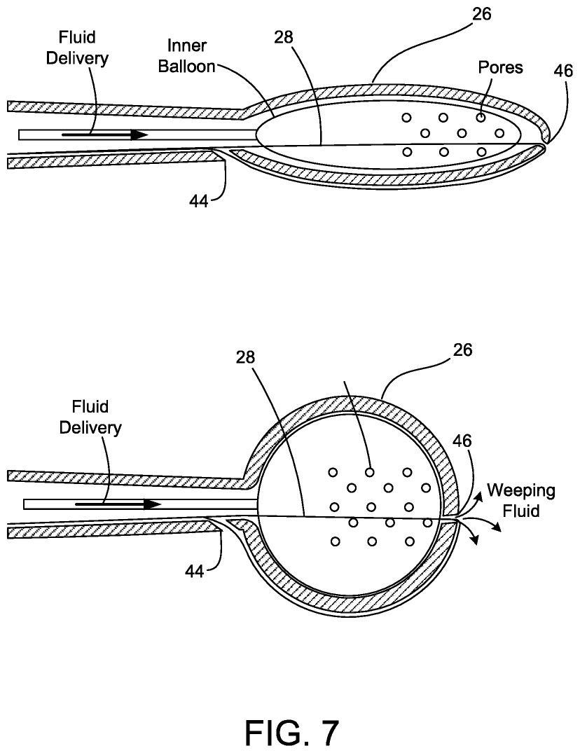

As shown in FIG. 9, for example, a conductive wire 28 passes within a lumen 64 of the hydrophilic insert 54 and along an external surface thereof. Similar to the conductive wires 28, the conductive wire 28 passing through the hydrophilic insert 54 is also electrically connected to the ablation generator 20. In some embodiments, the conductive member 28 is also configured to deploy the hydrophilic insert 54 from a delivery configuration to a deployed configuration (e.g., deployed as shown). For example, during use, the conductive member 28 can also contract or expand the hydrophilic insert 54 to modify the saline fluid flow as desired. For example, a control wire 58 may pass within the lumen of the tip 16, and may be grouped with other control wires (not shown) into a control line 56 that extends through the device lumen alongside the fluid delivery line 38. The control wires 58 can be connected to the conductive members 28 by a conductive link 60.

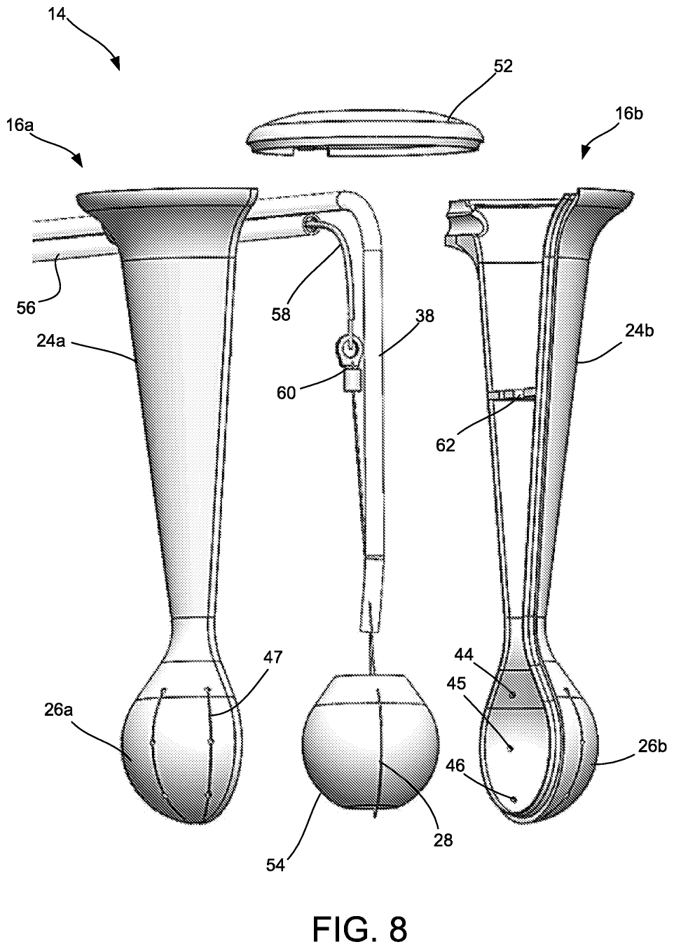

FIGS. 10A-10E are perspective views of a distal tip of the ablation device of FIG. 1 illustrating various electrode array configurations. In addition, while the conductive wires 28 have been described as extending along an external surface of the distal tip 16 in a direction that is parallel to the longitudinal axis of the device (as shown in a longitudinal configuration of conductive wires 28a in FIG. 10A), other configurations are possible. For example, one or more conductive wires 28b could extend along the external surface of the distal tip 16 in a direction that is perpendicular to the longitudinal axis of the device (as shown in a circumferential configuration in FIG. 10B). In other examples, one or more conductive wires 28c can extend from along the external surface of the distal tip 16 at an angle (e.g., non-parallel to the longitudinal axis of the device), as shown in an angled configuration in FIG. 10C. One or more conductive wires 28d, 28e, and 28f can also form a pattern along the external surface in which the conductive wires extend in various directions, as shown in a combined configuration in FIG. 10D. Additionally or alternatively, one or more conductive wires 28g can extend a reduced length of the external surface an alternative configuration in FIG. 10E.

While various conductive wires 28 have generally been described such that individual conductive members are energized or that the desired combination of conductive members is energized for a pre-selected or desired duration, in some cases, the desired combination of conductive members can be based on desired contact region of the distal tip 16.

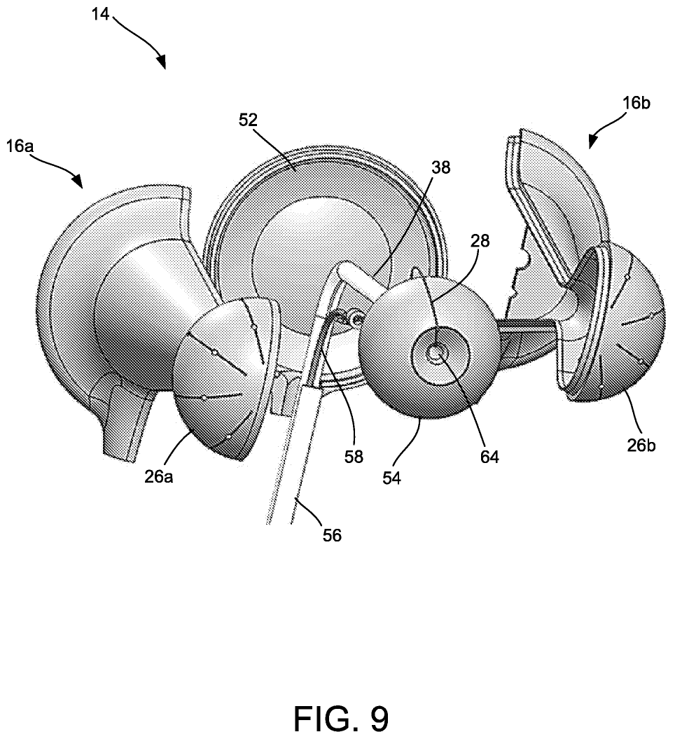

FIG. 11 is a side view of the distal tip 16 of the ablation device 14 of FIG. 1 including several clinical axes or sides. Each clinical axis or side includes one or more independently connected electrodes, which enables differential function and current independent drives and/or measurements. For example, referring to FIG. 11, the distal tip 16 can be divided into clinical axes or sides 66, 68, 70, 72, 74, and 75 (not shown). In other words, the distal tip 16 may include six clinical axes or sides of the distal portion (e.g., four sides or quadrants around spheroid body 70, 72, 74, and 75, and a top axis/side 66, and a bottom axis/side 68).

In other embodiments, where the distal tip 16 is a hemispherical body (i.e., one half of the spheroid body), as described in greater detail herein, the distal tip 16 can be divided into two sides, one side 72 being the convex portion of the hemispherical body, and the other side 70 being the planar side, with a top axis/side 66 and a bottom axis side 68. In such embodiments, the conductive wires 28 as described herein, only extend along the convex external surface of the distal tip 16 in a direction that is parallel to the longitudinal axis of the device in the configurations as described throughout.

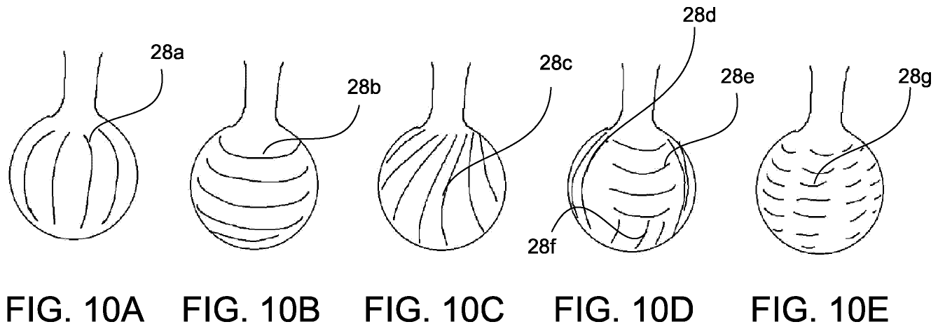

FIGS. 12A-12D are side and perspective views of the distal tip of the application device illustrating the different clinical axes or sides of FIG. 9. As shown in FIGS. 12A-12D, each clinical axis can include multiple independently connected conductive wires. For example, clinical axis/side 66 can include three independently connected conductive wires 76, clinical axis/side 68 can include three independently connected conductive wires 78, clinical axis/side 70 can include three independently controlled conductive wires 80, clinical axis/side 72 can include three independently connected conductive wires 82, clinical axis/side 74 can include three independently controlled conductive wires 84, and clinical axis/side 75 can include three independently controlled conductive wires 86. The independently connected conductive wires within each clinical axis or side allows for differential function and independent energy delivery and/or measurements. While FIGS. 12A-12D generally show three conductive wires for each clinical axis or side, other combinations are possible. For example, each of the clinical axes or sides can include a combination of conductive wires ranging from one conductive wire to ten or more conductive members.

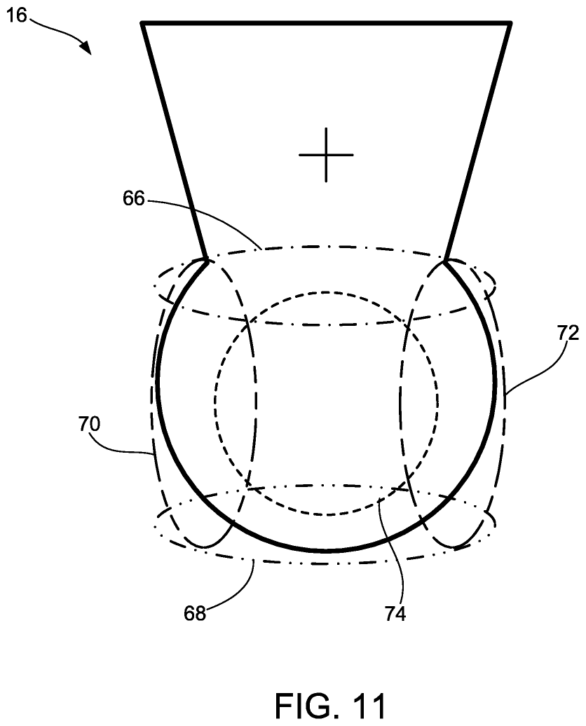

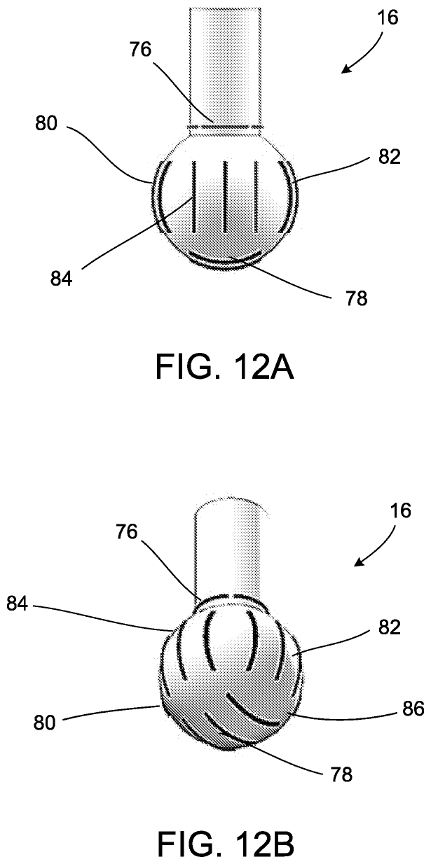

FIGS. 13 and 14 are perspective and exploded perspective views, respectively, of one embodiment of a device controller 19 consistent with the present disclosure. As shown, the controller 19 may include a first halve or shell 88a and a second halve or shell 88b for housing a PC board 90 within, the PC board 90 comprising circuitry and hardware for controlling various parameters of the device 14 during an ablation procedure. The controller 19 further includes a display 92, such as an LCD or LED display for providing a visual representation of one or more parameters associated with the device 14, including, but not limited to, device status (e.g., power on/off, ablation on/off, fluid delivery on/off) as well as one or more parameters associated with the RF ablation (e.g., energy output, elapsed time, timer, temperature, conductivity, etc.). The controller 19 may further include a top membrane 94 affixed over the PC board 92 and configured to provide user input (by way of buttons or other controls) with which a user (e.g., surgeon or medical professional) may interact with a user interface provided on the display 92. The controller 19 may be configured to control at least the amount of electrical current applied to one or more of the conductive wires 28 from the ablation generator 20 and the amount of fluid to be delivered to the device 14 from the irrigation pump/drip 22.

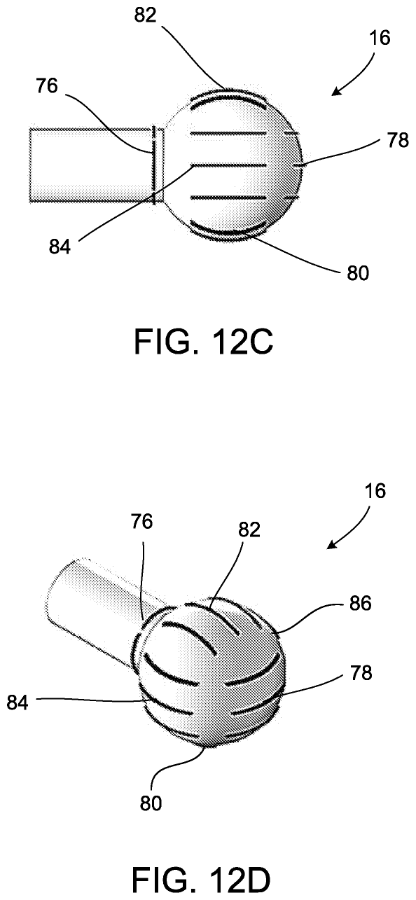

FIG. 15 is an exploded perspective view of another embodiment of an ablation device 14a consistent with the present disclosure. The device 14a is similarly configured as device 14 illustrated in FIG. 8, and includes similar elements. For example, the device 14a includes the distal tip 16 formed from two or more pieces (tip halves 16a and 16b) configured to be coupled to one another to form the unitary distal tip 16. Each half 16a and 16b includes cooperating neck portions 24a, 24b and spheroid bodies 26a, 26b, as well as a cap 52 to be coupled to both halves 16a and 16b so as to fully enclose the interior of the distal tip 16. As further illustrated, an electrical line 34 may be provided for coupling the conductive wires 28 to the controller 18 and ablation generator 20 and a fluid line 38 may be provided for providing a fluid connection between the irrigation pump or drip 22 to the distal tip 16 so as to provide a conductive fluid (e.g., saline) to the tip 16. The electrical line 34 and/or the fluid delivery line 38 can be supported by a stabilizing element 62 within the device lumen. In some cases, the stabilizing element 62 may be integral with the neck 24 of the distal tip 16.

The device 14a is configured to provide RF ablation via a virtual electrode arrangement, which includes distribution of a fluid along an exterior surface of the distal tip 16 and, upon activation of the electrode array, the fluid may carry, or otherwise promote, energy emitted from the electrode array to the surrounding tissue. For example, the nonconductive spheroid body 26 includes an interior chamber (when the first and second halves 26a, 26b are coupled to one another) for retaining at least a spacing member 96 (also referred to herein as “spacer ball”) and one or more hydrophilic inserts 98a, 98b surrounding the spacing member 96. The interior chamber of the distal tip 16 is configured to receive and retain a fluid (e.g., saline) therein from a fluid source. The hydrophilic inserts 98a, 98b are configured receive and evenly distribute the fluid through the distal tip 16 by wicking the saline against gravity. The hydrophilic inserts 98a and 98b can be formed from a hydrophilic foam material (e.g., hydrophilic polyurethane).

As previously described, the distal tip 16 may generally include a plurality of ports or apertures configured to allow the fluid to pass therethrough, or weep, from the interior chamber to an external surface of the distal tip 16. Accordingly, in some embodiments, all of the ports (e.g., proximal ports 44, medial ports 45, and distal ports 46) may be configured to allow for passage of fluid from the inserts 98a, 98b to the exterior surface of the distal tip 16. However, in some embodiments, only the medial ports 45 may allow for fluid passage, while the proximal and distal ports 44, 46 may be blocked via a heat shrink or other occlusive material.

The spacer member 96 may formed from a nonconductive material and may be shaped and sized so as to maintain the hydrophilic inserts 98a, 98b in sufficient contact with the interior surface of the distal tip wall, and specifically in contact with the one or more ports, such that the hydrophilic inserts 98a, 98b provides uniformity of saline distribution to the ports. In some embodiments, the spacer member 96 may have a generally spherical body, corresponding to the interior contour of the chamber of the spheroid body 26.

Accordingly, upon positioning the distal tip 16 within a target site (e.g., tissue cavity to be ablated), the electrode array can be activated and fluid delivery can be initiated. The fluid weeping through the ports to the exterior surface of the distal tip is able to carry energy from electrode array, thereby creating a virtual electrode. Accordingly, upon the fluid weeping through the port, a pool or thin film of fluid is formed on the exterior surface of the distal portion and is configured to ablate surrounding tissue via the RF energy carried from the electrode array.

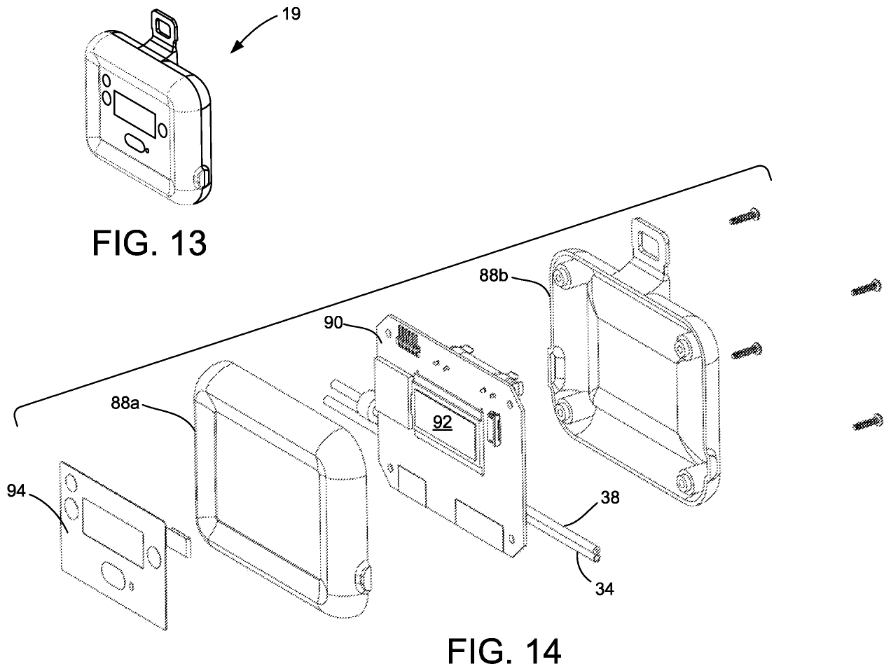

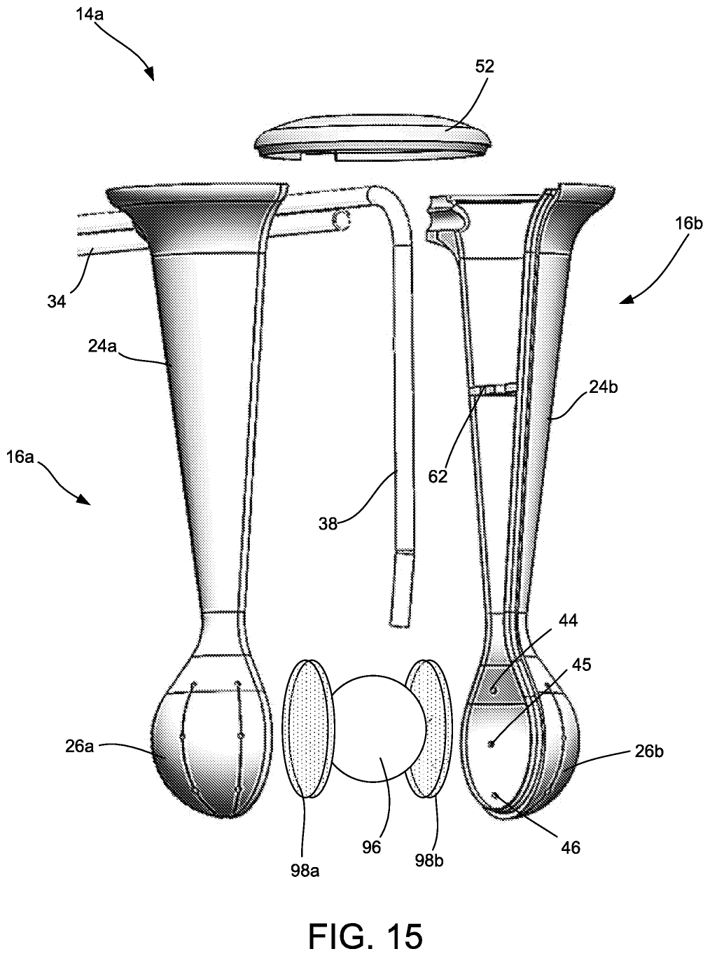

As previously described herein, conductive wires 28 may generally extend through a first port (e.g., the distal port 44), run along an external surface of the spheroid body 26 before re-entering the lumen of the distal tip 16 through another port (e.g., the proximal port 46). FIGS. 16, 17, 18A-18B, and 19A-19B illustrate another arrangement of conductive wires 28, in which at least four different conductive wires are provided, two of which serve as supply electrodes and the other two serve as return electrodes. Each of the four different conductive wires generally pass through at least two different proximal ports and two different distal ports, while remaining isolated from one another. FIG. 16 is a plan view of the ablation device 14a illustrating the two halves of the device tip 16a, 16b separated from one another and showing the external surface each, while FIG. 17 shows the interior surface of each.

FIGS. 18A and 18B are enlarged views of the spheroid body of the first halve 16a of the device 14a showing the exterior and interior surfaces, respectively, and further illustrating the particular arrangement of first and second conductive wires 28(1) and 28(2), partly in phantom, extending through proximal and distal ports 44, 46 of the spheroid body 26a. The following description of the first and second conductive wires 28(1) and 28(2) provides a general pathway of each wire, including passages through ports and extensions along lengths of the interior and exterior surfaces of the tip 16. In the illustrated embodiment, a first conductive wire 28(1) may serve as a return electrode while a second conductive wire 28(2) may serve as a supply electrode.

As shown, the first conductive wire 28(1) extends within the lumen of the tip 16a and passes through proximal port 44(1), extends along the exterior surface of the spheroid body 26a towards the distal ports (generally parallel to longitudinal axis of device), passes through distal port 46(1), extends along the interior surface of the body 26a towards adjacent distal ports (generally transverse to longitudinal axis of the device), passes through distal port 46(2), extends along the exterior surface of the spheroid body 26a back towards the proximal ports, passes through proximal port 44(2), extends along the interior surface of body 26a towards adjacent proximal ports, passes through proximal port 44(5), extends along the exterior surface of the spheroid body 26a back towards the distal ports, passes through distal port 46(5), extends along the interior surface of the body 26a towards adjacent distal ports, passes through distal port 46(6), extends along the exterior surface of the spheroid body 26a back towards the proximal ports, passes through proximal port 44(6), and extends back through lumen of the tip 16a. Accordingly, the first conductive wire 28(1) has at least four portions that extend along the exterior surface of the spheroid body 26a.

The second conductive wire 28(2) extends within the lumen of the tip 16a and passes through distal port 44(3), extends along the exterior surface of the spheroid body 26a towards the distal ports (generally parallel to longitudinal axis of device), passes through distal port 46(3), extends along the interior surface of the body 26a towards adjacent distal ports (generally transverse to longitudinal axis of the device), passes through distal port 46(4), extends along the exterior surface of the spheroid body 26a back towards the proximal ports, passes through proximal port 44(4), and extends back through lumen of the tip 16a. Accordingly, the second conductive wire 28(2) has at least two portions that extend along the exterior surface of the spheroid body 26a.

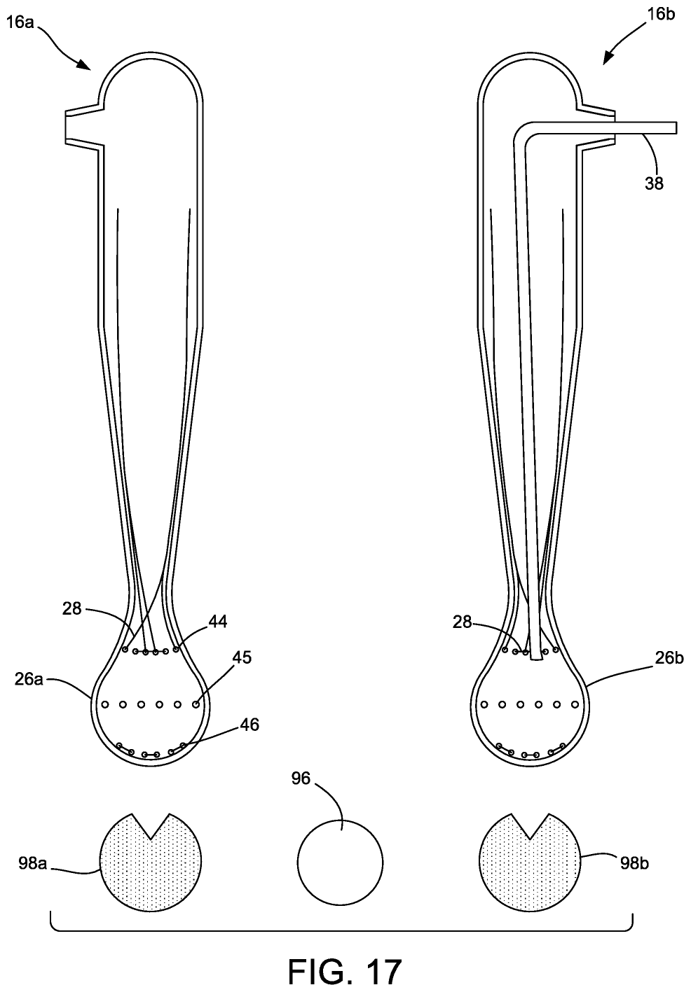

FIGS. 19A and 19B are enlarged views of the spheroid body of the second halve 16b of the device 14a showing the exterior and interior surfaces, respectively, and further illustrating the particular arrangement of third and fourth conductive wires 28(3) and 28(4) extending through proximal and distal ports of the spheroid body 26b. The following description of the third and fourth conductive wires 28(3) and 28(4) provides a general pathway of each wire, including passages through ports and extensions along lengths of the interior and exterior surfaces of the tip 16. In the illustrated embodiment, a third conductive wire 28(3) may serve as a return electrode while a second conductive wire 28(4) may serve as a supply electrode.

As shown, the third conductive wire 28(3) extends within the lumen of the tip 16a and passes through proximal port 44(9), extends along the exterior surface of the spheroid body 26b towards the distal ports (generally parallel to longitudinal axis of device), passes through distal port 46(9), extends along the interior surface of the body 26b towards adjacent distal ports (generally transverse to longitudinal axis of the device), passes through distal port 46(10), extends along the exterior surface of the spheroid body 26b back towards the proximal ports, passes through proximal port 44(10), and extends back through lumen of the tip 16a. Accordingly, the third conductive wire 28(3) has at least two portions that extend along the exterior surface of the spheroid body 26b.

The fourth conductive wire 28(4) extends within the lumen of the tip 16b and passes through proximal port 44(7), extends along the exterior surface of the spheroid body 26b towards the distal ports (generally parallel to longitudinal axis of device), passes through distal port 46(7), extends along the interior surface of the body 26b towards adjacent distal ports (generally transverse to longitudinal axis of the device), passes through distal port 46(8), extends along the exterior surface of the spheroid body 26b back towards the proximal ports, passes through proximal port 44(8), extends along the interior surface of body 26b towards adjacent proximal ports, passes through proximal port 44(11), extends along the exterior surface of the spheroid body 26b back towards the distal ports, passes through distal port 46(11), extends along the interior surface of the body 26b towards adjacent distal ports, passes through distal port 46(12), extends along the exterior surface of the spheroid body 26b back towards the proximal ports, passes through proximal port 44(12), and extends back through lumen of the tip 16a. Accordingly, the fourth conductive wire 28(4) has at least four portions that extend along the exterior surface of the spheroid body 26b.

Furthermore, each of the four conductive wires 28(1)-28(4) remain electrically isolated and independent from one another such that, each, or one or more sets of a combination of, the conductive wires, can independently receive an electrical current from the ablation generator and independently conduct energy, the energy including RF energy. This allows energy to be selectively delivered to a designated conductive wire or combination of conductive wires. This design also enables the ablation device to function in a bipolar mode because a first conductive wire (or combination of conductive wires) can deliver energy to the surrounding tissue through its electrical connection with an ablation generator while a second conductive wire (or combination of conductive wires) can function as a ground or neutral conductive member.

The independent control of each wire or sets of wires allows for activation (e.g., emission of RF energy) of corresponding portions of the electrode array. For example, the electrode array may be partitioned into specific portions which may correspond to clinical axes or sides of the distal portion of the device. In one embodiment, the electrode array may include at least four distinct portions (i.e., individual or sets of conductive wires) corresponding to four clinical axes or sides of the distal portion (e.g., four sides or quadrants around spheroid body).

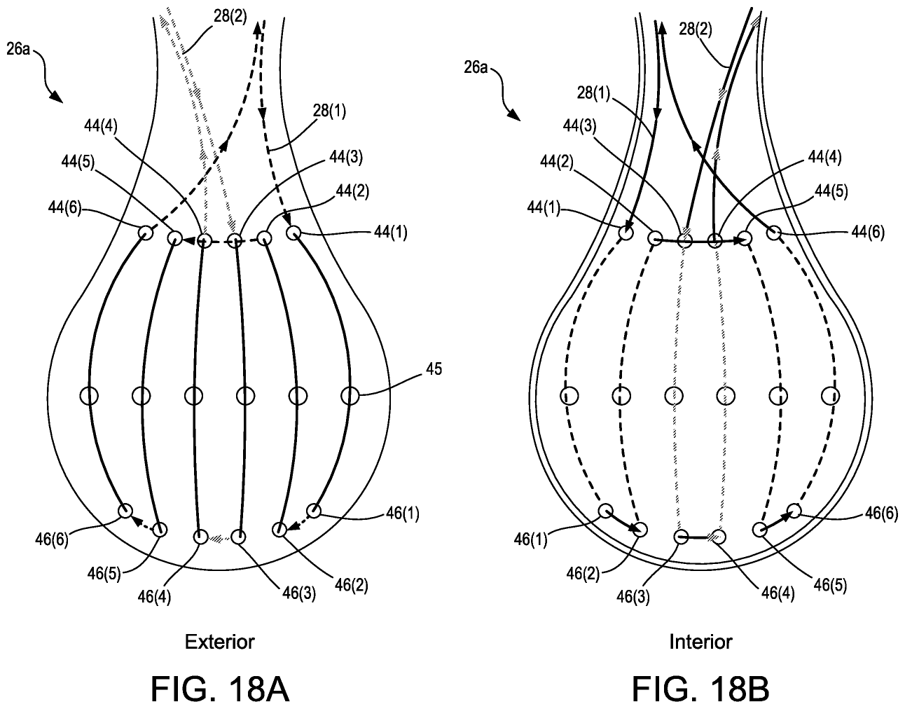

FIG. 20 is a schematic illustration of the ablation device 14a illustrating delivery of fluid from the irrigation pump 22, as controlled by the controller 19, to the hydrophilic inserts 98a, 98b within the interior chamber of the distal tip 16, wherein the fluid can be subsequently distributed to an exterior surface of the spheroid body 26 resulting in a virtual electrode arrangement upon activation of one or more portions of the electrode array. As shown, the saline may be distributed through at least the medial ports 45, such that the weeping saline is able to carry electrical current from electrode array, such that energy is transmitted from the electrode array to the tissue by way of the saline weeping from the ports, thereby creating a virtual electrode. Accordingly, upon the fluid weeping through the medial port, a pool or thin film of fluid is formed on the exterior surface of the spheroid body 26 and is configured to ablate surrounding tissue via the electrical current carried from the electrode array.

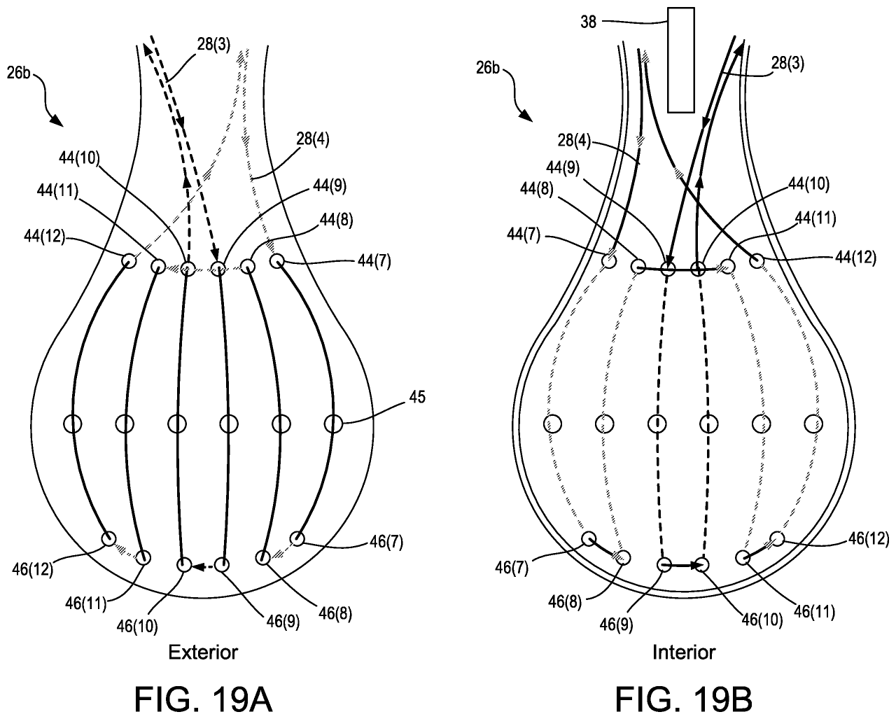

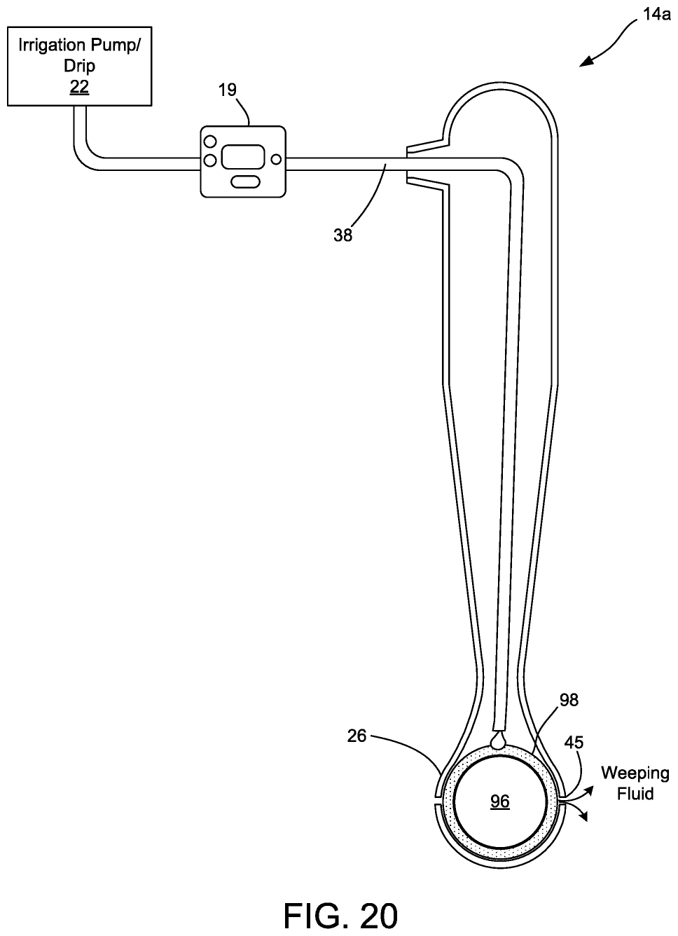

FIGS. 21 and 22 are perspective and plan views of a detachable mount 100 for holding and maintaining a temperature probe 102 (or any other separate monitoring device) at a desired position, as indicated by arrow 106, relative to the spheroid body 26 of the distal tip of the ablation device 14. In particular, the mount 100 allows for an operator (e.g., surgeon) to releasably couple a temperature probe 102, or other measurement device, to the ablation device 14a and further position the working end 104 of the probe 102 in close proximity to the spheroid body 2 for the collection of temperature data during an RF ablation procedure.

As previously described herein, the controller 18, 19 may be configured to provide a surgeon with the ability to control ablation, such as controlling the supply of power to one or more conductive wires as well as control the delivery of fluid to the device tip 16. Furthermore, the controller 18, 19 may provide device status (e.g., power on/off, ablation on/off, fluid delivery on/off) as well as one or more parameters associated with the RF ablation (e.g., energy output, elapsed time, timer, temperature, conductivity, etc.). Thus, in some instances, it may be important to monitor at least the temperature adjacent to the device tip 16 during the ablation procedure, as well as pre-ablation and post-ablation, as temperature may be indicative of the status of surrounding tissue that is being, or is intended to be, ablated. Furthermore, it may be important to monitor the temperature at certain distances from the device tip 14 and at certain angles. Current devices may include a thermocouple mechanism integrated into the device. However, such configurations lack the ability to obtain temperature measurement at specific distances and angles relative to the ablation tip. The mount 100 is configured to provide a surgeon with the ability to adjacent the angle at which the temperature probe is positioned relative to the device tip 16 as well as the distance from the device tip 16, thereby overcoming the drawbacks of integrated thermocouples.

As shown, the mount 100 generally includes a body having a first end 108 configured to be releasably coupled to at least the proximal end of the device 14 by way of a clamping mechanism or latch-type engagement. The first end 108 includes a top guard member 110 configured to partially enclose at least the proximal end of the device 14, to further enhance securement of the mount 100 to the device 14. The mount 100 further includes an arm member 112 extending from the first end 108 and providing a second end 114 positioned a distance from the first end 108. The second end 114 is configured to hold the temperature probe 102 at a desired position, including a desired distance from the spheroid body 26 and a desired angle θ relative to the longitudinal axis of the ablation device. For example, in one embodiment, the second end 114 may include a bore or channel configured to receive and retain a portion of the temperature probe 102 within. The second end 114 may further allow for the temperature probe 102 to translate along the bore or channel, as indicated by arrow 116, to thereby adjust the distance of the temperature probe tip 104 relative to the spheroid body of the device tip. In some embodiments, the arm 112 and/or second end 114 may articulate relative to one another and/or the first end 108. Accordingly, the angle of the temperature probe 102 may also be adjusted as desired.

Accordingly, a tissue ablation devices, particularly the applicator heads described herein, may be well suited for treating hollow body cavities, such as cavities in breast tissue created by a lumpectomy procedure. The devices, systems, and methods of the present disclosure can help to ensure that all microscopic disease in the local environment has been treated. This is especially true in the treatment of tumors that have a tendency to recur.

FIG. 23 is a perspective view of the distal portion or tip 16 of another embodiment of an ablation device 14b consistent with the present disclosure. The distal tip 16 may include a neck portion 24 and a generally hemispherical body 27 (i.e., one-half of the spheroid body), extending distally from the neck 24. The device 14b is similarly configured as device 14a illustrated in FIG. 15, and includes similar elements. As previously described herein, the tip 16 of the ablation device 14 further includes an electrode array positioned thereon. The electrode array includes at least one conductive member 28. The plurality of conductive members 28 extend within the distal tip 16 along a convex external surface of the spheroid body 26 and may be configured in any way as described herein. Similarly, the electrode array may further include one or more stabilizing members 30 configured to provide support for the plurality of conductive wires 28.

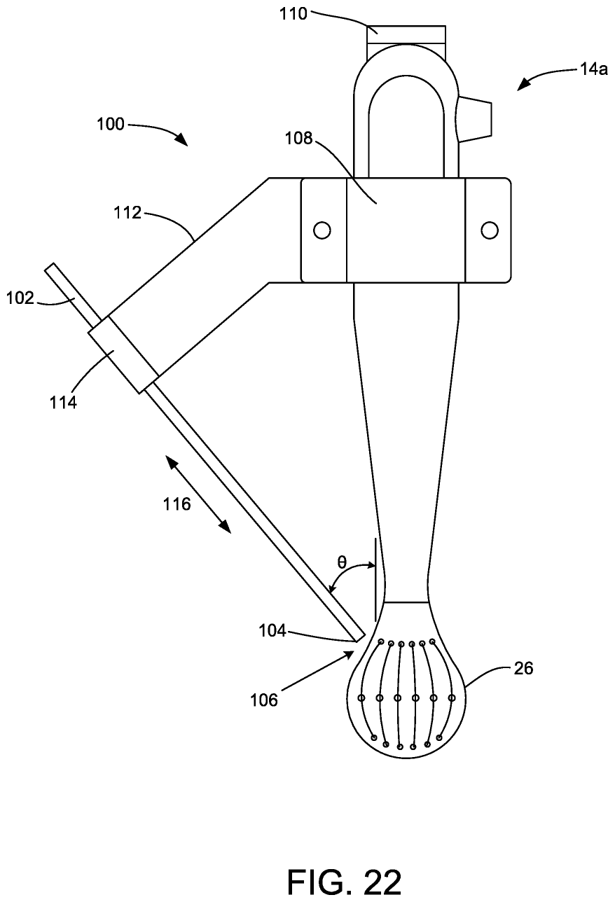

FIG. 24 is an exploded perspective view of an ablation device 14b consistent with the present disclosure. For example, the device 14b includes the distal tip 16 formed by two or more pieces (tip halves 16a and 16b) configured to be coupled to one another to form the unitary distal tip 16. The device 14b includes a hemispherical body 27 (i.e., one-half of the spheroid body). As further illustrated, conductive wires 28 may generally extend through a first port (e.g., a distal port 44), run along a convex exterior surface 120 of the rigid hemispherical body 27 (i.e., the external surface of one half of the spheroid body 26b) before re-entering the lumen of the distal tip 16 through another port (e.g., a proximal port 46).

The device 14b includes the distal tip 16 formed from two or more pieces (tip halves 16a and 16b) configured to be coupled to one another to form the unitary distal tip 16. Each half 16a and 16b includes at least cooperating neck portions 24a, 24b. The device 14b includes a hemispherical body 27 formed by at least one solid flat body 27a and one convex body 27b (i.e., one half of a spheroid body, 26b) configured so that the solid flat body 27a is coupled with the convex body 27b to form the hemispherical body 27. Both halves 16a and 16b are configured so as to fully enclose the interior of the distal tip 16.

As further illustrated, an electrical line 34 may be provided for coupling the conductive wires 28 to the controller 18 and ablation generator 20 and a fluid line 38 (not shown) may be provided for providing a fluid connection between the irrigation pump or drip 22 to the distal tip 16 so as to provide a conductive fluid (e.g., saline) to the tip 16. The electrical line 34 and/or the fluid delivery line 38 can be supported by a stabilizing element 62 within the device lumen. In some cases, the stabilizing element 62 may be integral with the neck 24 of the distal tip 16. For example, the nonconductive hemispherical body 27 includes an interior chamber (when the first and second halves 27a, 27b are coupled to one another) for retaining a hemispherical spacing member 96a (i.e., a half of the spacing member 96 described throughout) and a hydrophilic insert 98 (i.e., either 98a or 98b as described throughout) surrounding the convex side of the spacing member 96a.

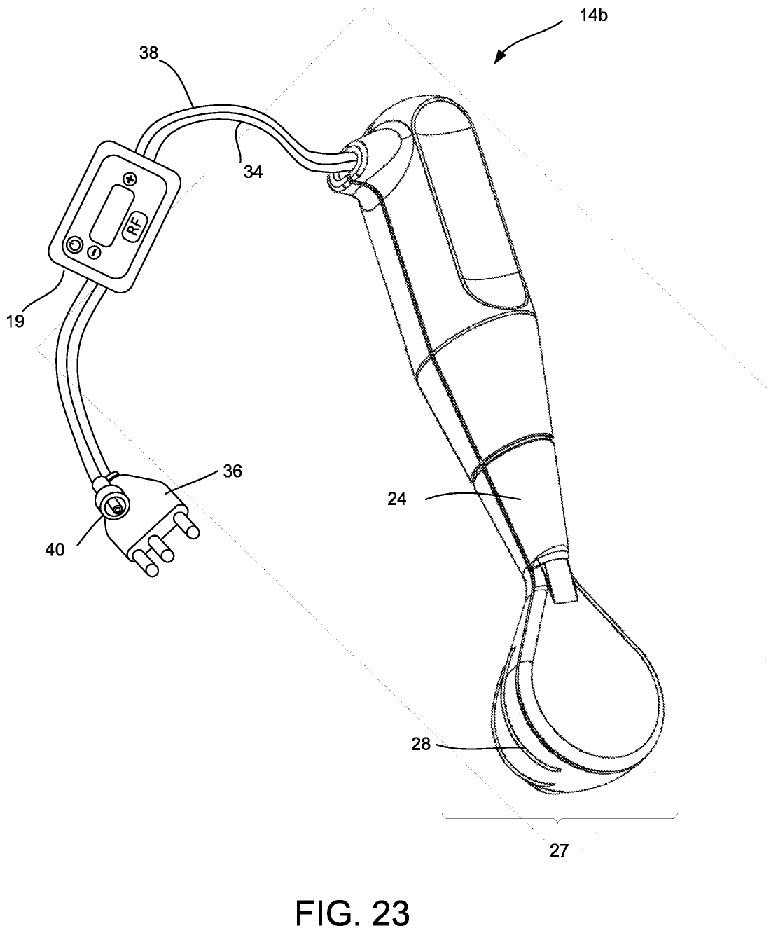

FIG. 25 is a plan view of the interior surface of an ablation device 14b. The nonconductive hemispherical body 27 includes an interior chamber (when the first and second halves 27a, 27b are coupled to one another) for retaining at least a hemispherical spacing member 96a and one or more hydrophilic inserts 98 surrounding the convex side of the spacing member 96a. The interior chamber of the distal tip 16 is configured to receive and retain a fluid (e.g., saline) therein from a fluid source within the device 14b. As described throughout, the hydrophilic insert 98 is configured to receive and evenly distribute the fluid through the distal tip 16 by wicking the saline against gravity. The convex side of the hemispherical body 27 may generally include a plurality of ports or apertures configured to allow the fluid to pass therethrough, or weep, from the interior chamber to a convex exterior surface of the distal tip 16. As similarly described throughout, all of the ports (e.g., proximal ports 44, medial ports 45, and distal ports 46) on the convex side may be configured to allow for passage of fluid from the insert 98 to the convex exterior surface 120 of the distal tip 16, or, in other embodiments, only the medial ports 45 may allow for fluid passage, while the proximal and distal ports 44, 46 may be blocked via a heat shrink or other occlusive material.

The hemispherical spacer member 96a may formed from a nonconductive material and may be shaped and sized so as to maintain the hydrophilic insert 98 in sufficient contact with the interior surface of the distal tip wall, and specifically in contact with the one or more ports, such that the hydrophilic insert 98 provides uniformity of saline distribution to the ports. In some embodiments, the spacer member 96a may have a generally hemispherical body, corresponding to the interior contour of the chamber of the hemispherical body 27.

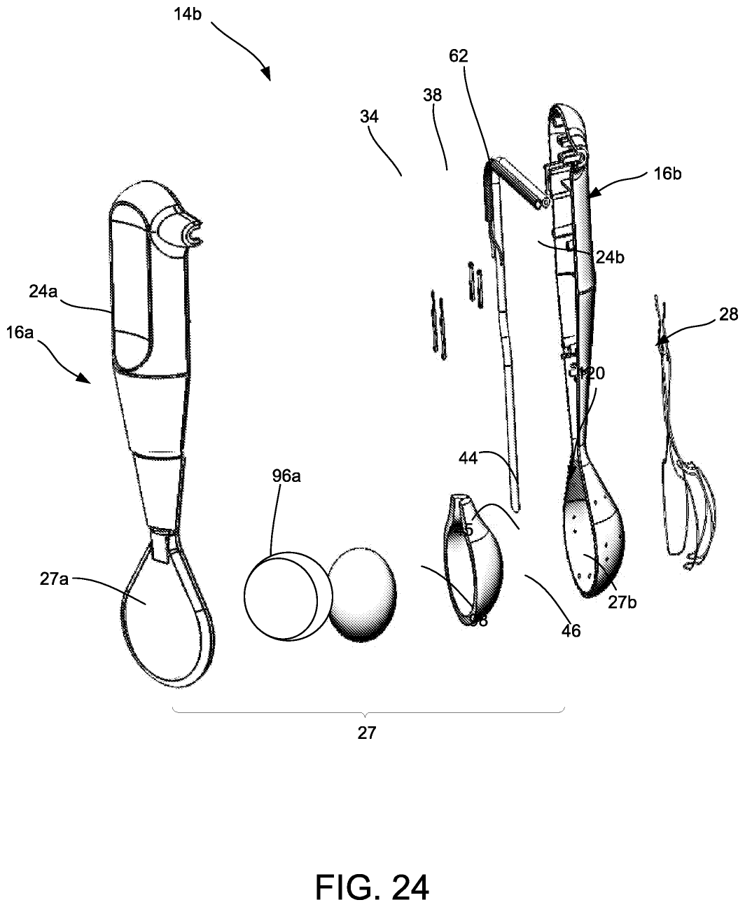

FIG. 26 is a plan view of the ablation device 14b illustrating the two halves of the device tip 16a, 16b separated from one another and showing the external surface each. A solid flat body 27a of the device 14b is coupled to the convex body 27b having conductive wires 28 which may generally extend through a first port (e.g., the distal port 44), run along a convex exterior surface 120 of the rigid hemispherical body 27 (i.e., the external surface of one half of the spheroid body 26b) before re-entering the lumen of the distal tip 16 through another port (e.g., the proximal port 46). The conductive wires 28 may be arranged along the convex exterior surface in any manner as described throughout. For example, FIGS. 19A-19B illustrate an arrangement of conductive wires 28, in which at least two different conductive wires are provided along the convex side of one-half of the spheroid body 26, one of which serves as a supply electrode and the other which serves as a return electrode.

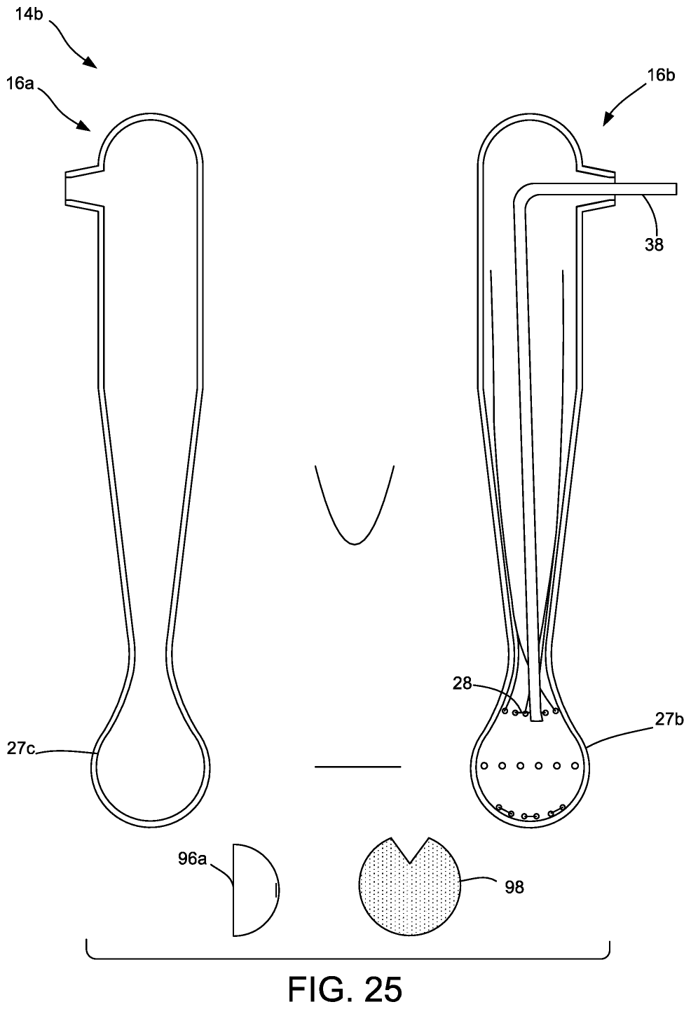

FIG. 27 is a perspective view of another embodiment of an ablation device 14c consistent with the present disclosure. The device 14c is similarly configured as device 14a illustrated in FIG. 15, and includes similar elements. For example, the device 14c includes the distal tip 16 formed by two or more pieces (tip halves 16a and 16b) configured to be coupled to one another to form the unitary distal tip 16. However, the device 14c includes a cavity 29 within its hemispherical body 27 (i.e., one-half of the spheroid body) capable of receiving and holding fluid from a user. The cavity 29 of the hemispherical body 27 includes receiving ports 124 for transporting fluid from the cavity into an interior cavity of the hemispherical body 27. As such, the device 14c as depicted lacks a fluid lumen. As further illustrated and as described throughout, conductive wires 28 may generally extend through a first port (e.g., a distal port 44), run along a convex exterior surface of the rigid hemispherical body 27 (i.e., the external surface of one half of the spheroid body 26b) before re-entering the lumen 34 of the distal tip 16 through another port (e.g., a proximal port 46).

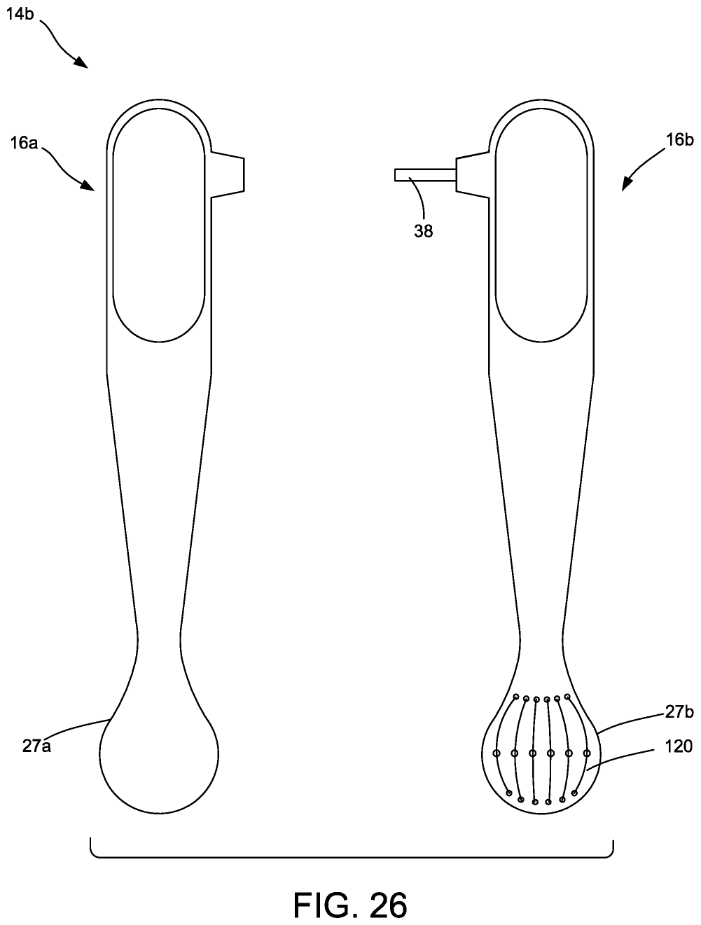

FIG. 28 is an exploded perspective view of an ablation device 14c consistent with the present disclosure. The device 14c includes the distal tip 16 formed from two or more pieces (tip halves 16a and 16b) configured to be coupled to one another to form the unitary distal tip 16. Each half 16a and 16b includes at least cooperating neck portions 24a, 24b. The device 14b includes a hemispherical body 27 formed by at least one concave body 27c and one convex body 27b (i.e., one half of a spheroid body, 26b) configured so that the concave body 27c nests within the convex body 27b to form the hemispherical body 27 having an exterior cavity 29. The neck portion 24a is configured to be coupled to the concave body 27c to form the tip half 16a. Both halves 16a and 16b are configured so as to fully enclose the interior of the distal tip 16.

As further illustrated, an electrical line 34 may be provided for coupling conductive wires 28 to the controller 18. The electrical line 34 can be supported by a stabilizing element 62 within the device lumen. In some cases, the stabilizing element 62 may be integral with the neck 24 of the distal tip 16. In some embodiments of the invention, instead of receiving the fluid via a device lumen, the fluid is received from an external source, such as from a user inputting (e.g., pouring) the fluid to the device cavity 29. As previously described, the distal tip 16 may also generally include a plurality of ports or apertures configured to allow the fluid to pass therethrough, or weep, from the interior chamber to an external surface of the distal tip 16. In some embodiments of the invention, some the plurality of ports or apertures may be configured to receive the fluid (i.e., receiving ports 124) and transport it into the interior chamber, before the fluid is allowed to weep through the ports configured to do so.

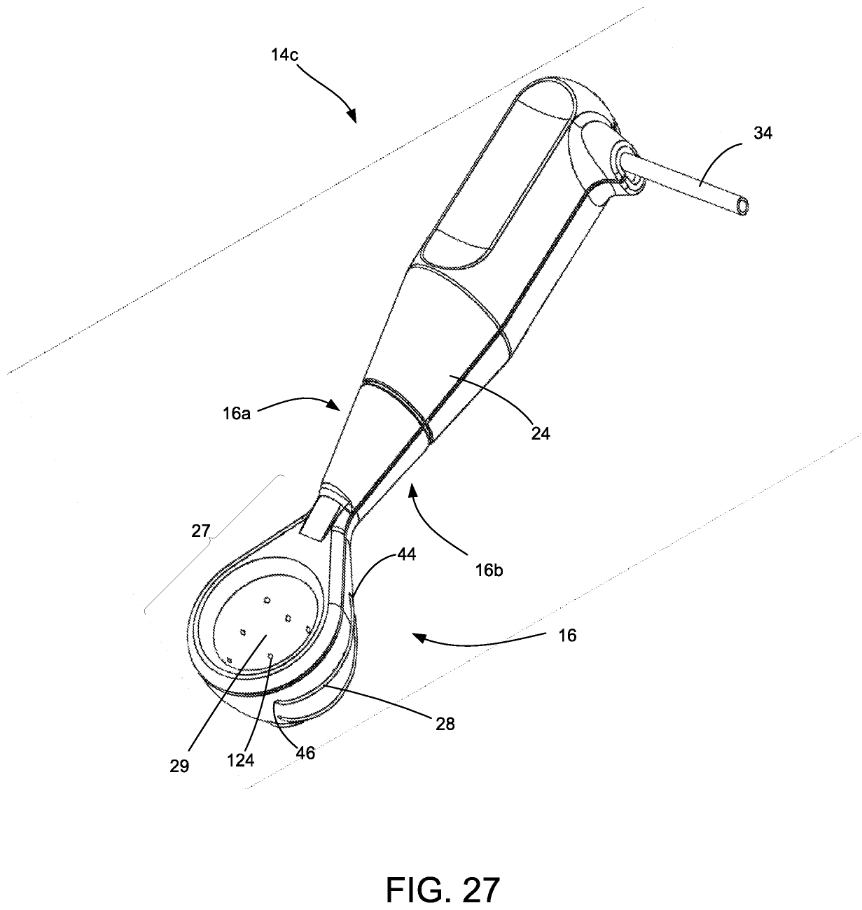

As illustrated, the device 14c is configured to receive a fluid via the cavity 29 and to provide RF ablation via a virtual electrode arrangement, which includes distribution of the fluid along a convex exterior surface 120 of the hemispherical body 27 of the distal tip 16 and, upon activation of the electrode array, the fluid may carry, or otherwise promote, energy emitted from the electrode array to the surrounding tissue. For example, the nonconductive hemispherical body 27 includes an interior chamber 122 (shown in FIG. 29) (formed when the concave body 27c and the convex body 27b are nested together) for retaining at least one or more hydrophilic inserts 98 corresponding to the interior contour of the hemispherical body 27. The interior chamber 122 of the distal tip 16 is configured to receive and retain a fluid (e.g., saline) therein from the cavity 29 through a plurality of receiving ports 124. The hydrophilic insert 98 is configured to receive and evenly distribute the fluid through the plurality of ports (e.g., proximal ports 44, medial ports 45, and distal ports 46) of the convex body 27b of the distal tip 16 by wicking the saline against gravity. The hydrophilic insert 98 can be formed from a hydrophilic foam material (e.g., hydrophilic polyurethane).

FIG. 29 is a cross-sectional view of the hemispherical body 27 of the ablation device 14b. The cavity 29 of the convex body 27c includes a plurality of receiving ports 124 which are configured to receive a fluid from the cavity 29 and to pass the fluid into the interior chamber 122. The convex body 27c is nested with the concave body 27b. As previously described, the distal tip 16 may also generally include a plurality of ports or apertures configured to allow the fluid to pass therethrough, or weep, from the interior chamber to an external surface of the distal tip 16. Accordingly, in some embodiments, the external surface is the convex exterior surface 120 of the hemispherical body 27 and all of the ports of the convex body 27b (e.g., proximal ports 44, medial ports 45, and distal ports 46) may be configured to allow for passage of fluid from the insert 98 to the convex exterior surface 120 of the distal tip 16. However, in some embodiments, only the medial ports 45 may allow for fluid passage, while the proximal and distal ports 44, 46 may be blocked via a heat shrink or other occlusive material.

The hydrophilic insert 98 is in sufficient contact with the surface of the interior chamber 122 of the distal tip wall, and specifically in contact with one or more of the receiving ports 124 and one or more of the ports of the convex body 27b (e.g., the medial ports 45), such that the hydrophilic insert 98 provides uniformity of saline distribution to the ports of the convex body 27b.

Accordingly, upon positioning the convex body 27b portion of the distal tip 16 near a target site (e.g., tissue to be ablated), fluid can be delivered to the cavity 29 and then the electrode array can be activated. The fluid weeping through the ports of the convex body 27b to the convex exterior surface 120 of the distal tip 16 is able to carry energy from electrode array, thereby creating a virtual electrode. Accordingly, upon the fluid weeping through the ports of the convex body 27b, a pool or thin film of fluid is formed on the convex exterior surface 120 of the distal tip 16 and is configured to ablate surrounding tissue via the RF energy carried from the electrode array.

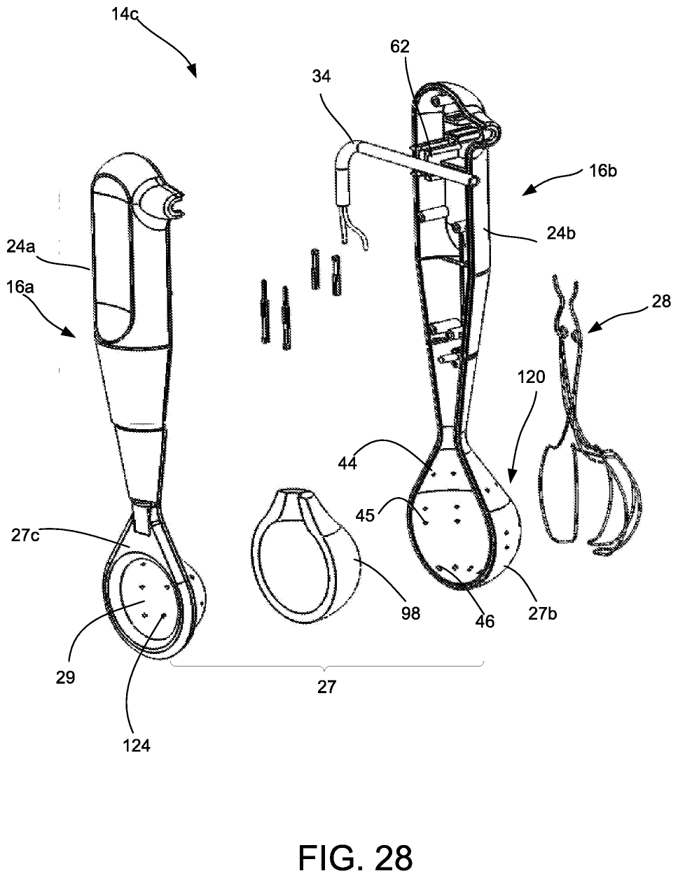

FIG. 30 is a plan view of the ablation device 14c illustrating the two halves of the device tip 16a, 16b separated from one another and showing the external surface of each. A cavity 29 for receiving fluid from a user having with a plurality of receiving ports 29 is present on the convex body 27c of the device 14c. Conductive wires 28 may generally extend through a first port (e.g., the distal port 44) on the convex body 27b and run along a convex exterior surface 120 of the rigid hemispherical body 27 (i.e., the external surface of one half of the spheroid body 26b) before re-entering the lumen of the distal tip 16 through another port (e.g., the proximal port 46). The conductive wires 28 may be arranged along the convex exterior surface in any manner as described throughout. For example, FIGS. 19A-19B illustrate an arrangement of conductive wires 28, in which at least two different conductive wires are provided along the convex side of one-half of the spheroid body 26, one of which serves as a supply electrode and the other which serves as a return electrode.

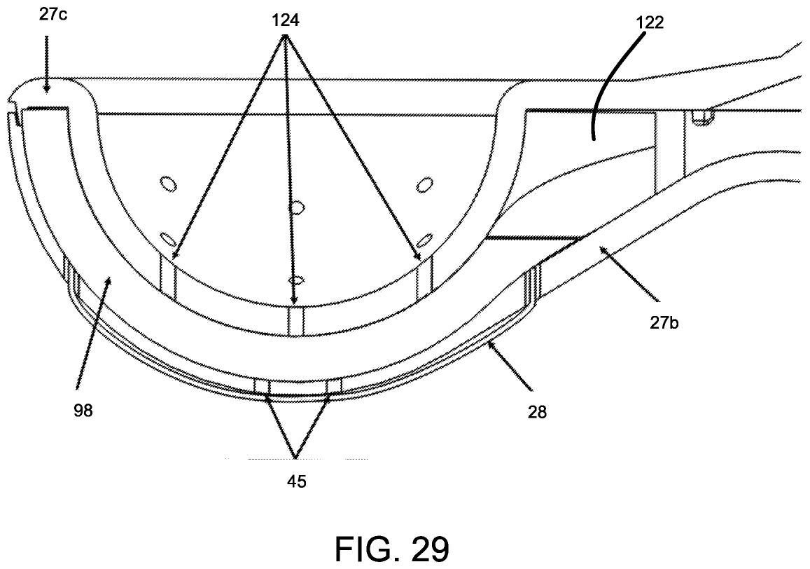

FIG. 31 is a perspective view of the device 14c illustrating the cavity 29 receiving fluid from an external source. Once in the cavity 29, the fluid passes through the receiving ports 29 into the interior chamber of the distal tip 16. The device 14c allows a user to control how much fluid is passed through the device by simply providing a means by which a user may add fluid to the cavity 29 as desired. As such, unlike some of the other embodiments of the invention, the device 14c does not require a complicated switch to control the delivery of fluid through the device, and instead is operated with a simple controller 18 known in the art (e.g., a foot pedal) to control the electrical connection and input to the device 14c.

As previously described, the distal portion of a treatment device consistent with the present disclosure may include various shapes and/or sizes depending on the specific treatment to be provided and/or the specific site to which the treatment is to be applied. For example, FIGS. 32A and 32B are top and side views, respectively, of an exemplary shape of the distal portion, notably the body 26, 27 of the device, illustrating a hemispherical shape. While illustrated as being a relatively true spherical shape, the body may also include an oblate spheroid shape or a prolate spheroid shape.

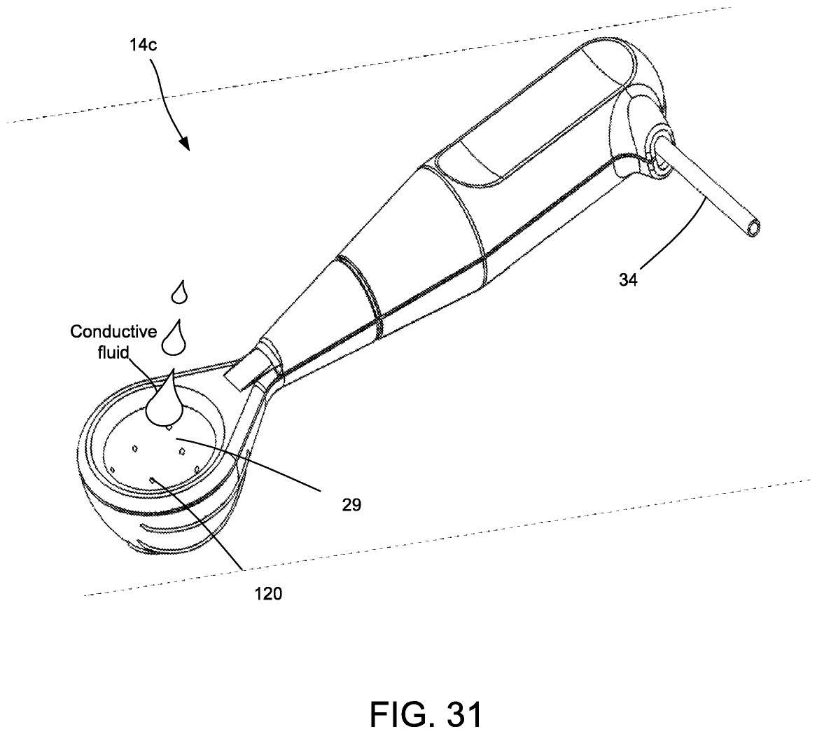

FIGS. 33A and 33B are top and side views, respectively, of another exemplary shape of the distal portion of the device, illustrating a hemiellipsoidal shape. As generally understood, an ellipsoid is a surface that may be obtained from a sphere by deforming it by means of directional scalings, or more generally, of an affine transformation. In particular, an ellipsoid is understood to be a quadric surface (i.e., a surface that may be defined as the zero set of a polynomial of degree two in three variables). An ellipsoid may generally include three pairwise perpendicular axes of symmetry which intersect at a center of symmetry, called the center of the ellipsoid. The line segments that are delimited on the axes of symmetry by the ellipsoid are called the principal axes, or simply axes of the ellipsoid. If the three axes have different lengths, the ellipsoid is said to be tri-axial or rarely scalene, and the axes are uniquely defined. If two of the axes have the same length, then the ellipsoid is an ellipsoid of revolution, also called a spheroid. In this case, the ellipsoid is invariant under a rotation around the third axis, and there are thus infinitely many ways of choosing the two perpendicular axes of the same length. If the third axis is shorter, the ellipsoid is an oblate spheroid; if it is longer, it is a prolate spheroid. If the three axes have the same length, the ellipsoid is a sphere.

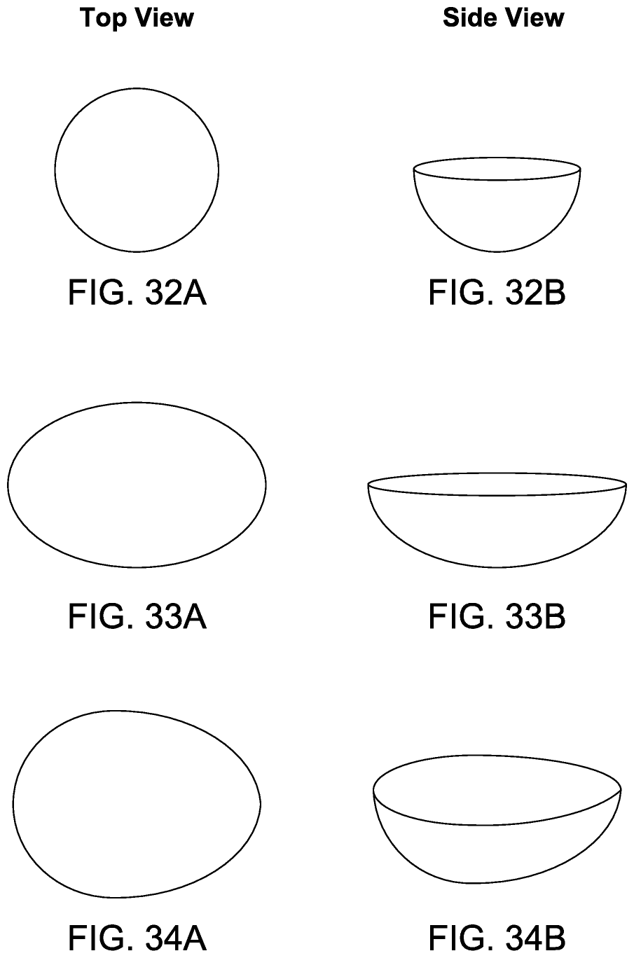

FIGS. 34A and 34B are top and side views, respectively, of one exemplary shape of the distal portion of the device, illustrating a hemiovoidal shape. In the illustrated embodiment, the hemiovoidal shape generally resembles the shape of a chicken egg, for example (i.e., only one axis of symmetry). However, the body 26, 27 may include other forms of ovals, such as, for example, a Cassini oval, Moss's Egg, Cartesian oval, or other forms.

It should further be noted that the shapes illustrated in FIGS. 32A through 34B are merely representative of the various forms that the distal portion or tip 16, notably body 26, 27 can take. In other embodiments, the body 26, 27 is not limited to arcuate shapes and rather may include any polygonal shape (i.e., triangular, square, rectangular, multi-sided, etc.).

The tissue ablation devices having hemispherical heads described herein are particularly useful for treating surface area tissue, such as resection tissue after removal of a tumor or a biopsy, or to treat surface lesions.

As used in any embodiment herein, the term “controller”, “module”, “subsystem”, or the like, may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The controller or subsystem may, collectively or individually, be embodied as circuitry that forms part of a larger system, for example, an integrated circuit (IC), system on-chip (SoC), desktop computers, laptop computers, tablet computers, servers, smart phones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

What is claimed is:

1. A medical device comprising:
a distal portion comprising a rigid arcuate body including a plurality of ports and an interior chamber; and
a conductive wire disposed along at least a portion of a convex exterior surface of the rigid arcuate body and configured to conduct energy to be carried by a conductive fluid passing from the interior chamber and through one or more of the plurality of ports.

2. The medical device of claim 1, wherein the rigid arcuate body includes an exterior cavity.

3. The medical device of claim 2, wherein the exterior cavity is configured to receive the conductive fluid and further to pass the fluid to the interior chamber through one or more of the plurality of ports.

4. The medical device of claim 3, wherein one or more of the plurality of ports is a receiving port within the exterior cavity and is configured to allow passage of the conductive fluid from the exterior cavity to the interior chamber.

5. The medical device of claim 2, wherein the distal portion is defined by a first halve and a second halve, wherein the first halve comprises a neck portion and a concave portion and the second halve comprises a neck portion and a convex portion, wherein the first halve is coupled to the second halve to form the distal portion comprising the rigid arcuate body defining the exterior cavity, the plurality of ports, and the interior chamber.

6. The medical device of claim 1, further comprising a hydrophilic insert disposed within the interior chamber, wherein the insert is configured to receive the conductive fluid and to distribute the conductive fluid to the convex exterior surface through one or more of the plurality of ports.

7. The medical device of claim 1, wherein one or more of the plurality of ports is configured to allow passage of the conductive fluid from the interior chamber to the convex exterior surface.

8. The medical device of claim 7, wherein, upon receipt of an electric current, the conductive wire is configured to conduct radiofrequency (RF) energy to be carried by the conductive fluid passing through one or more of the plurality ports to the convex exterior surface for ablation of a tissue.

9. The medical device of claim 7, wherein the plurality of ports comprises one or more medial ports for allowing passage of the conductive fluid to the convex exterior surface.

10. The medical device of claim 1, wherein the conductive wire axially translates along a longitudinal axis of the device.

11. The medical device of claim 1, wherein the conductive wire is substantially aligned with at least one of the plurality of ports on the convex exterior surface.

12. The medical device of claim 1, wherein the plurality of ports comprises a plurality of proximal ports and distal ports on the convex exterior surface, wherein the conductive wire passes through at least one of the proximal ports and through a corresponding one of the distal ports such that a portion of the conductive wire has a length that extends along the convex exterior surface of the distal portion between the corresponding proximal and distal ports.

13. The medical device of claim 12, wherein the conductive wire is one of a plurality of conductive wires, each of the plurality of conductive wires is disposed along at least a portion of the convex exterior surface of the distal portion.

14. The medical device of claim 13, wherein each of the plurality of conductive wires passes through at least one of the proximal ports and through a corresponding one of the distal ports, wherein each of the plurality of proximal ports corresponds to a separate one of the plurality of distal ports such that a portion of a conductive wire passing through a set of corresponding proximal and distal ports has a length that extends along the convex exterior surface of the distal portion between the corresponding proximal and distal ports.

15. The medical device of claim 14, wherein the plurality of ports comprises one or more medial ports for allowing passage of the conductive fluid to the convex exterior surface.

16. The medical device of claim 15, wherein each conductive wire is substantially aligned with a respective one of the medial ports.

17. The medical device of claim 13, wherein each of the plurality of conductive wires, or one or more sets of a combination of conductive wires, is configured to independently conduct energy to be carried by a conductive fluid passing from the interior chamber and through one or more of the plurality of ports for ablation of a target tissue.

18. The medical device of claim 1, further comprising a handle including an electrical line to deliver electric current to the conductive wire, wherein the electrical line is in electrical communication with an electrical connection.

19. The medical device of claim 18, wherein the handle further comprises a lumen for receiving the conductive fluid, wherein the lumen is in fluid communication with the interior chamber of the rigid arcuate body.

20. The medical device of claim 1, wherein the rigid arcuate body comprises a nonconductive material.

21. The medical device of claim 1, wherein the arcuate body comprises a hemispherical shape.

22. The medical device of claim 1, wherein the arcuate body comprises a hemiellipsoidal shape.

23. The medical device of claim 1, wherein the arcuate body comprises a hemiovoidal shape.
